(19) United States
(12) Patent Application Publication
(10) Pub. No.: US 2026/0029401 A1
(43) Pub. Date: Jan. 29, 2026

(54) METHODS AND SYSTEMS FOR CHARACTERIZING PROTEOFORMS OF SIGNIFICANT PROTEINS OF INTEREST

(71) Applicant: Nautilus Subsidiary, Inc., Seattle, WA (US)

(72) Inventors: Parag MALLICK, San Mateo, CA (US); Andreas HUHMER, Redwood City, CA (US); Kara JUNEAU, San Carlos, CA (US); Vivekananda BUDAMAGUNTA, San Carlos, CA (US); Grant NAPIER, San Carlos, CA (US)

(21) Appl. No.: 19/280,018

(22) Filed: Jul. 24, 2025

Related U.S. Application Data
(60) Provisional application No. 63/676,145, filed on Jul. 26, 2024.
(60) Provisional application No. 63/687,689, filed on Aug. 27, 2024.
(60) Provisional application No. 63/709,289, filed on Oct. 18, 2024.
(60) Provisional application No. 63/761,547, filed on Feb. 21, 2025.
(60) Provisional application No. 63/779,692, filed on Mar. 28, 2025.
(60) Provisional application No. 63/827,592, filed on Jun. 20, 2025.

(51) Int. Cl. G01N 33/573; G01N 33/58; G01N 33/68; G01N 33/74

(52) U.S. Cl. CPC G01N 33/573; G01N 33/582; G01N 33/68; G01N 33/74; G01N 2333/71; G01N 2333/912

(57) ABSTRACT

Methods, reagents, kits and systems for analyzing different proteoforms of proteins of interest are provided. The provided methods, systems, etc. provide detection, characterization and quantitation of proteoforms for different biologically relevant proteins for monitoring and characterizing biological processes.

### RELATED APPLICATIONS

This application claims priority to each of Provisional U.S. Patent Application No. 63/676,145, filed on Jul. 26, 2024, Provisional U.S. Patent Application No. 63/687,689, filed on Aug. 27, 2024, Provisional U.S. Patent Application No. 63/709,289, filed on Oct. 18, 2024, Provisional U.S. Patent Application No. 63/761,547, filed Feb. 21, 2025, Provisional U.S. Patent Application No. 63/779,692, filed Mar. 28, 2025, and Provisional U.S. Patent Application No. 63/827,592, filed Jun. 20, 2025, the full disclosures of which are hereby incorporated herein by reference in their entirety for all purposes.

### BACKGROUND

Biological researchers are constantly seeking better ways to look into the functions of living things, in order to understand the keys to life and health, the causes of disease and dysfunction, and to help identify possible paths of intervention or influence to achieve better outcomes for all of these.

High throughput, highly sensitive detection and analysis technologies have given rise to great advances in the field of biological research. For example, medical research and clinical diagnostics have seen significant advances resulting from the emergence of high throughput technology platforms that routinely decode the human genome or human transcriptome in a matter of hours. An individual's genome, as a blueprint for the components of a given biological system, can provide some insights into development, behavior, risk of disease, responsiveness to therapeutic treatments, longevity and many other characteristics. As such, the genome can provide a powerful source for evaluating risk and predicting outcomes to certain treatments or medications.

Likewise, an individual's transcriptome is the collection of RNA transcripts that are expressed from the genome. The RNA transcripts are, in turn, translated into proteins which may, in some cases be further modified post translationally. The proteins function as the workhorses that perform the biological functions in biological systems as instructed by the genome. In some cases, characterization and quantification of the transcriptome can lead to clinically relevant diagnoses or prognoses for a given biological system, e.g., a patient.

The advent of high-throughput, relatively inexpensive and routine genetic analysis tools and processes has made genomic or transcriptomic analysis a convenient starting point in looking at biological functions. Unfortunately, however, these analyses are really directed at proxies for actual biological function. The genome, for example, is a snapshot of a blueprint, in many cases, taken at conception, that provides very little insight into the present functioning of a biological system. The transcriptome, on the other hand, provides a more contemporaneous measure of that biological function, but still falls short of actual biological operations beyond a measure of what genes are transcribed and when. The information provided, again, is removed from the actual biological functions being carried out at any given moment in time within the biological system, and as a result, in many cases, provides inadequate diagnostic or prognostic precision to guide treatment.

To gain more insightful views into the function, dysfunction and manipulation of biological systems, researchers need analytical systems and methods that measure the actual biological operations that are occurring within these biological systems, including looking at the presence, prevalence, flux and function of the various proteins within those systems. The set of proteins present within a given biological system is generally referred to as the proteome of that system.

While identifying and quantifying the various proteins in a biological system at any given time potentially yields significant amounts of information as to the functioning of that system, protein presence, absence or quantity alone are not the only key pieces of information. In particular, many proteins within a given proteome function differently, are removed from the system, or engage in or cause myriad different interactions based upon the particular form of the protein that exists. In particular, proteins may be subjected to post translational modifications that result in phosphorylation, glycosylation, truncation, aggregation, or other modifications that can alter the proteins' function(s), subcellular location, degradation or post translational cleavage, longevity or how they interact with other aspects of the system. Similarly, pre-translation modifications to proteins, such as splice variants, that may include excised portions of transcribable genes, can yield proteins that differ from full-length gene products, and as a result, function differently. Any given protein species may exist as different molecules that are each modified in a potentially large number of different ways. The collection of these various forms of a given protein within a given proteome are generally referred to as the different proteoforms of that protein. And across a given proteome, tens, hundreds, thousands or more proteins may each exist as different proteoforms. Scientists are just beginning to gain understanding of how different proteoforms can produce dramatically different outcomes within biological systems. For example, differentially phosphorylated versions of the microtubule-associated protein tau (or “Tau”, for short), which generally functions to stabilize the structure of neurons in the brain, has been associated with the formation of amyloid plaques in the brain tissue of patients suffering from Alzheimer's Disease, and is believed to play a key role in progression of the disease (See, e.g., U.S. patent application Ser. No. ______, filed of even date herewith (Attorney Docket No. 0095-US-1). Likewise, differentially modified versions of alpha-synuclein protein (α-syn) have similarly been implicated as potential participants in the onset and progression of Parkinson's Disease (See, e.g., Magalhaes and Lashuel, NPJ Parkinsons's Disease (2022)8:93; and U.S. patent application Ser. No. ______, filed of even date herewith (Attorney Docket No. 0104-US-1), the full disclosures of which are incorporated herein by reference in their entirety for all purposes.

Accordingly, it is highly desirable to provide methods, systems and reagents for use in accurately and sensitively characterizing and quantifying a variety of different proteoforms within the proteomes of biological systems, and particularly those implicated in specific diseases like Parkinson's. Unfortunately, many existing technologies for analyzing proteins, such as protein or peptide sequencing technologies, mass spectrometry methods, and the like, lack the ability to both comprehensively characterize and quantify proteoforms at high throughput and high sensitivity needed to broadly understand the full proteoform landscape of the disease, its role in onset and progression of the disease, and its implications for diagnosis and prognosis for development and progression of the disease in patients. The present disclosure addresses these and many other needs.

### SUMMARY

Described herein are improved methods, processes, systems, components, and reagents useful in analyzing proteoforms from biological samples. These improvements yield more sensitive, reproducible analysis of proteoforms of a variety of different proteoforms of proteins of interest, e.g., proteins and proteoforms that are of biological relevance/interest in biological research, diagnostics and therapeutics.

Generally speaking, provided herein are methods, processes, systems, devices and reagents that are useful in characterizing different proteoforms of proteins of interest in a variety of different pathologies and critical biological functions, including, for example, catenin beta-1, mitogen-activated protein kinase 1, Epidermal growth factor receptor, Leucin rich repeat serine/threonine-protein kinase 2, HER2, RAC-alpha serine/threonine-protein kinase, and mothers against decapentaplegic homolog 2 proteins. These methods, processes, systems, devices and reagents may exploit individually assessable proteins including the proteins of interest that may be individually interrogated using affinity reagents specific for one or more characteristics of different proteoforms of the proteins of interest, and identifying those proteins of interest which possess such characteristics based upon the binding of such affinity reagents. The different proteoforms are then characterized based upon the different proteoform characteristics that are identified.

The methods, processes, systems, reagents and devices described herein may be employed in everything from analyzing the presence, absence and/or relative abundance of different proteoforms in a sample, as well as analyzing, identifying, characterizing and/or quantifying different sets of proteoforms of particular proteins of interest in a sample to provide proteoform profiles of such samples, which may, in turn, be used to compare among samples to evaluate changes in those profiles as a function of key parameters, such as between healthy and disease associated tissues, over time to evaluate disease onset and/or progression and order of biological events leading to the same, response to treatments or to potential effectors of biologics associated with the disease pathology, and the like.

In accordance with certain aspects, provided herein are methods of analyzing proteins in a first sample. These methods typically comprise providing a population of individual protein molecules from the sample wherein the individual protein molecules are individually addressable, and wherein the population of individual molecules comprises a plurality of individual molecules of at least one protein selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2). Aproteoform of the protein of interest represented by each of the plurality of individual molecules of at least one protein of interest is identified based upon the identification of the presence or absence of at least 3 different modifications within each of the individual molecules of the protein of interest. A plurality of proteoforms of the at least one protein of interest present in the sample is then characterized.

In other aspects, provided herein are systems for characterizing proteins that comprise one or more solid supports comprising molecules of at least one protein of interest immobilized thereon, wherein the protein of interest is selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2) proteins, and wherein individual molecules of the at least one protein of interest are individually addressable. The systems also typically include a source of a plurality of different affinity reagents, each different affinity reagent having a binding affinity to the at least one protein of interest having a different modification, as well as a fluidic system for delivering the plurality of different affinity reagents to the one or more solid supports to contact the affinity reagents with the individual molecules of the at least one protein of interest. Additionally, the systems typically comprise a detector for detecting whether each of the different affinity reagents binds to individual molecules of the at least one protein of interest, and a processor programed to characterize proteoforms of the at least one protein of interest present on the one or more solid supports from detected binding or nonbinding of the different affinity reagents to the individual molecules of the at least one protein of interest.

In still other aspects, provided herein are arrays that comprise a plurality of individual molecules of at least one protein of interest deposited on a surface of the array and positioned to be individually addressable, wherein the at least one protein of interest is selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2), and wherein, and wherein the plurality of molecules of the at least one protein of interest comprise at least two proteoforms of the protein of interest. The arrays, typically also include, in at least one configuration, a first affinity reagent having binding specificity for at least a first characteristic of at least one of the two proteoforms of at least one protein of interest, the first affinity reagent being bound to individual molecules of at least one protein of interest possessing the first characteristic of at least one of the two proteoforms of at least one protein of interest.

In still further aspects, provided herein are libraries of reagents that comprise a plurality of sources of affinity reagents, where each source of the plurality of sources contains a separate affinity reagent, and wherein each affinity reagent comprises (i) a binding specificity for a different characteristic of one or more proteoforms of at least one protein of interest selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2), and (ii) a detectable label attached to the affinity reagent.

### DESCRIPTION OF THE FIGURES

FIG. 1 schematically illustrates a protein analysis process and system.

FIG. 2 provides a high-level overview of a proteoform analysis and quantification approach.

FIG. 3 illustrates certain modification sites for different proteoforms and isoforms of the catenin beta 1 protein.

FIG. 4 illustrates certain modification sites for different proteoforms and isoforms of the ERK2 protein.

FIG. 5 illustrates certain modification sites for different proteoforms and isoforms of the EGFR protein.

FIG. 6 illustrates certain modification sites for different proteoforms and isoforms of the HER2 protein.

FIG. 7 illustrates certain modification sites for different proteoforms and isoforms of the LRRK2 protein.

FIG. 8 illustrates certain modification sites for different proteoforms and isoforms of the AKT1 protein.

FIG. 9 illustrates certain modification sites for different proteoforms and isoforms of the SMAD2 protein.

FIG. 10 schematically illustrates a system and its component parts, for use in carrying out the methods and processes described herein.

FIG. 11 schematically illustrates an exemplary proteoform characterization for a hypothetical protein having a series of phosphorylation sites that are differentially phosphorylated in the different proteoforms of the protein (Panel A) and their relative abundance (Panel B).

### DETAILED DESCRIPTION

### I. General

Provided herein are methods, reagents, systems and processes for use in analyzing and characterizing proteoforms from biological samples. Proteoforms typically refer to the potential various states of a given protein or set of proteins within a biological system, where such states may be defined by one or more of transcriptional or translational modifications or variations in such protein, and/or post translational modifications made to such proteins, including such modifications as post translational cleavage, degradation, phosphorylation, aggregation, acetylation, glycosylation (e.g., N and O linked glycosylation), amidation, nitration, hydroxylation, methylation, ubiquitylation, sulfation, or any of a host of additional alkylation, acylation, lipidation, disulfide, iodination amino acid addition, or other modifications made to protein molecules or their constituent amino acid side chains or terminal groups. Within a sample, a particular protein species may exist in multiple different proteoforms, i.e., having different modifications or patterns of modifications.

The general methods, processes, systems, devices and reagents described herein have been described for use in identifying and characterizing proteoforms of a different types of proteins, including for example, the microtubule protein Tau, or in identifying and/or characterizing the proteoforms of tau protein from biological samples (See, U.S. Provisional Patent Application No. 63/676,145, filed Jul. 26, 2024, U.S. Provisional Patent Application No. 63/687,689, filed Aug. 27, 2024, U.S. Provisional Patent Application No. 63/709,289, filed Oct. 18, 2024, and U.S. Provisional Patent Application No. 63/761,547, filed Feb. 21, 2025, and U.S. patent application Ser. No. ______, filed of even date herewith (Atty Docket No. 0095-US-1), the full disclosures of which are hereby incorporated herein by reference in their entirety for all purposes) and alpha-synuclein protein and its proteoforms (See, e.g., U.S. Provisional Patent Application No. 63/779,692, filed Mar. 28, 2025, and U.S. patent application Ser. No. ______, filed of even date herewith (Atty Docket No. 0104-US-1).

As described herein, the proteoform analysis methods, processes, systems, devices and reagents noted above are particularly suited to characterization of a variety of different modified or differentially created or processed versions of a number of proteins of significance in biological samples, including in particular, catenin beta-1, mitogen-activated protein kinase 1, epidermal growth factor receptor, Leucin rich repeat serine/threonine-protein kinase 2, HER2, RAC-alpha serine/threonine-protein kinase, and mothers against decapentaplegic homolog 2 proteins, and for elucidation of more comprehensive views of their proteoform and/or isoform make-up of biological samples at, e.g., different stages of pathology onset and progression associated with such proteins, as well as from healthy samples, or samples from patients that have yet to exhibit physiological symptoms of these pathologies.

In accordance with the methods described herein, in certain cases, analysis of proteoforms begins with the isolation of individual protein or polypeptide molecules in a manner that allows for their individual interrogation and analysis at the single molecule level. In particular, by analyzing individual, intact or undigested protein molecules of a proteoform, one can more accurately identify which proteoforms are present within a given sample, as well as provide relative quantification of those proteoforms in that sample.

In general, individual protein molecules within a sample may be isolated by immobilizing them on a solid support. In some cases, this may include isolation of an individual protein molecule of a sample on a bead or particle that may be individually interrogated and analyzed, while in other cases, individual protein molecules may be immobilized on different locations in a solid surface of an array, such that the different locations may be individually interrogated and separately analyzed.

One example of an array-based approach for protein analysis uses the approach described in, e.g., U.S. Pat. Nos. 10,473,654B1, 11,545,234B1, and Eggertson, et al. bioRxiv, the full disclosures of which are hereby incorporated herein by reference in their entirety for all purposes, where individual protein molecules are coupled to the surface of an array in separate, optically resolvable locations. The individual proteins are then iteratively probed using detectable affinity reagents that bind to identifiable traits of the proteins, such as specific compositional components, e.g., specific amino acid sequences or sequence contexts. These bound affinity reagents may then be detected, indicating the presence of that particular identifiable trait in the protein or polypeptide that is immobilized at that location.

For example, in the general proteome analysis methods described herein, affinity reagents used are capable of binding to small subunits of the proteins, like trimers or tetramer epitopes (3 or 4 amino acid segments) or other short or small sequence contexts of the protein. These reagents are iteratively contacted with the immobilized proteins on the array surface under conditions where affinity binding can occur. Once the reagents bind to proteins on the array and background reagents are washed away, the bound affinity reagents may be detected, typically through a detectable label group associated with the affinity reagent, such as a fluorophore. Binding of the labeled affinity reagent at a given location on the array indicates the likely presence of the particular epitope in the protein at that location. By iteratively probing using different affinity reagents, and assessing the probability associated with the binding events, one can potentially identify each protein that exists at each spot on the array. Moreover, by using affinity reagents that are not highly specific for an individual protein, but instead are capable of binding larger subsets of the proteome, e.g., multiple proteins containing a given trimer or tetramer epitope, one can potentially deconvolute a very large number of different proteins using a comparatively small number of affinity reagents. This “protein identification by short epitope mapping” (or “prism”) approach is described in detail in U.S. Pat. Nos. 10,473,654B1, 11,545,234B1, and Eggertson, et al. bioRxiv, previously incorporated herein by reference.

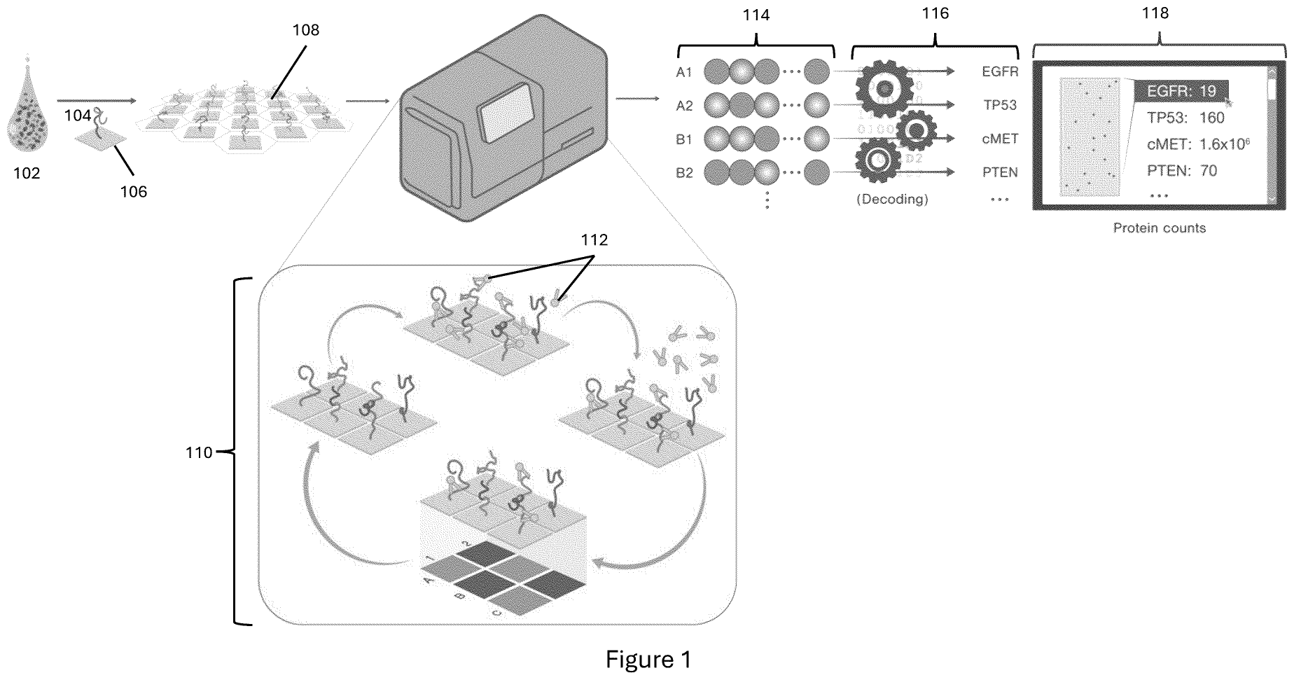

FIG. 1 illustrates a high-level overview of a process used for characterizing large numbers of proteins in a sample using the Prism approach described above. As shown, a protein containing sample 102 is obtained for analysis. Samples for analysis may be derived from any of a wide variety of biological systems, including animal, plant, microbial, viral, or the like. In some cases, model systems may be used to derive samples, such as genetically modified model murine or other mammalian systems that are engineered to exhibit certain disease traits or phenotypes, organoid models, e.g., engineered 3D immune-glial-neurovascular human multicellular integrated brains (miBrain). Other samples may be derived from past or present patients, and taken from, e.g., human tissue samples, blood samples, and/or biopsies. Moreover, samples may be derived from any of a variety of sources within a particular organism. For example, for animal derived samples, samples may be obtained from tissue, e.g. as cells or cell lysates, organs, organoids, blood or plasma, or cerebrospinal fluids, or any other sources that may have protein profiles of biological interest.

In the context of an array-based approach for analysis, proteins in the sample are treated to attach individual protein molecules 104 to individual particles, such as beads or structured nucleic acid particles or SNAPs 106. Once coupled to their respective SNAPs, the individual protein molecules are deposited and immobilized upon the surface of an array 108, where the SNAPs' size and/or surface binding characteristics result in the individual protein molecules being sufficiently spaced apart that they can be analyzed separately upon the surface of the array. For ease of illustration, arrays are shown with relatively small numbers of isolated proteins. However, it will be appreciated that an array surface may have upwards of 10s of thousands to 100s of thousands, to millions to billions of locations at which individual protein or polypeptide molecules may be located and separately interrogated/detected, e.g., 10,000 or more individual polypeptides, 100,000, or more individual polypeptides, 1,000,000 or more individual polypeptides, 10,000,00 or more individual polypeptides, 100,000,000 or more individual polypeptides, 1,000,000,000 or more individual polypeptides, or even 10,000,000,000 or more individual polypeptides on the surface of the arrays. Examples of this process and the resulting arrays are described in detail in, for example, U.S. Pat. Nos. 11,603,383B1, 11,505,795B1, WO 2023/102336A1, and Aksel et al., bioRxiv, the full disclosures of which are hereby incorporated herein by reference in their entirety for all purposes.

As discussed elsewhere herein, because the arrays described herein are comprised of individually addressable molecules of proteins, and in particular, the proteins of interest, they will generally reflect the dynamic ranges of molecules described elsewhere herein, e.g., from 1 to 9 orders of magnitude in relative concentration, which means that an array could include a single molecule f a given proteoform of a protein of interest, while also including 100s, thousands, 10s of thousands, hundreds of thousands, millions or even billions of other molecules, including other proteoforms of the same protein of interest.

Once created, an array of individual protein molecules may be iteratively interrogated (shown in panel 110) with affinity reagents 112 that are capable of binding to relatively short epitopes within the proteins, e.g., trimer, tetramers or other short sequence contexts of amino acids. In certain aspects, such interrogation is carried out iteratively with individual or limited sets of affinity reagents being contacted with the surface of the array 108. As noted previously, by utilizing affinity reagents that may bind to multiple proteins, but not all proteins, one can iteratively narrow down the identity of a protein molecule at any given position based upon the pattern of affinity reagents that binds to the protein at that location. As a result, one may be able to identify tens of thousands of proteins with a far smaller number of affinity reagents than if one were to use only highly specific affinity reagents, e.g., affinity reagents that specifically bind to only one protein. Again, examples of this analytical approach are described in, for example, U.S. Pat. Nos. 10,473,654B1, 11,545,234B1, and Eggertson, et al. bioRxiv, previously incorporated herein by reference.

In process, separate interrogation steps introduce different affinity reagents, or mixtures of affinity reagents, to the surface of the array, as shown in the expanded panel. These reagents are typically labeled, e.g., with fluorescent dyes, so that they may be detected. Following an incubation step to allow affinity reagents to bind to their specific target epitopes, excess reagents are washed away and the surface of the array is scanned using a fluorescence detection system, e.g., a scanning fluorescence microscope, and those points on the array where the affinity reagents are bound are detected and recorded. In some cases, different affinity reagents may carry differently detectable labels, e.g., fluorescent labels having different emission spectra, so as to allow simultaneous interrogation with 2, 3, 4 or more different affinity reagents. In these cases, the detection system will typically include optics, e.g., filters and directional components, that separate and separately measure signals having different spectral characteristics, thus allowing separate detection of the different affinity reagents bound to the array at the same time. Alternatively or additionally, different probes may be differentially detectable based upon their differing characteristics, e.g., their binding kinetics to target proteins or epitopes, such that one can differentiate two probes binding to the same protein molecule based upon the kinetics of the binding interaction, e.g., on and/or off rates. In such cases, real time observation optics may be employed to monitor binding and release of different affinity probes over time.

Following interrogation and scanning (whether in multiple rounds or fewer, the pattern of where different reagents did and did not bind (schematically illustrated at 114, are used to decode which proteins are at which positions on the array. These decoding processes typically utilize probability models (e.g., as schematically represented at 116) to assess the likelihood of true and false positive and negative binding events to ultimately identify individual proteins. At the end of the process, the identities and quantities of each type of protein on the surface of the array may then be determined (as shown at 118), and ultimately extrapolated back to the identity and quantity of different proteins within the sample. Although described in terms of iterative interrogation with individual affinity reagents for ease of understanding, it will be appreciated that interrogation steps may utilize multiple affinity reagents that are capable of separate detection, despite being present in the same analysis. For example, multiple different affinity reagents may be labeled with differentially detectable labels, e.g., fluorescent labels having different emission spectra, fluorescent lifetimes, etc. such that one may differentially detect binding of the different affinity reagents to proteins on the array.

### II. Proteoform Analysis

### A. Proteoform Characterization

In the context of proteoform analysis, however, the methods described herein seek to identify which proteoforms of particular proteins exist within the sample. Thus, in addition to being able to identify where and how often a particular type of protein is located on the array, and thus, within a sample, using the methods described herein, one can additionally or alternatively identify which proteoform of that protein is present in each location on the array, and thus in the sample from which the array was created. Additional context for the methods, processes, reagents, and systems described herein may be found in published U.S. Patent Application No. 2022/0236282, International Patent Application Nos. PCT/US24/15132, and WO 2023/038859, the full disclosures of which are hereby incorporated herein by reference in their entirety for all purposes.

For example, in some cases, proteoforms of a particular protein may exist as differently phosphorylated proteins within the same sample, meaning that different proteins may be phosphorylated at different amino acid residues in the protein, and may additionally be phosphorylated at one or more potential phosphorylation sites within the protein. By probing the array (including individually located molecules of the particular protein of interest) with multiple affinity probes that specifically recognize different phosphorylated species of the proteins of interest, e.g., recognizing and binding to the phosphorylated version of a particular epitope within the protein of interest, one can identify which phosphorylated epitopes, if any, are co-located on the array with the proteins of interest. Moreover, since multiple different probing events are carried out for the different phosphorylation sites in such proteins, one can determine the pattern of phosphorylation of each molecule of the protein of interest on the array, e.g., if and where in a protein's amino acid sequence a protein molecule of interest may be phosphorylated. Lastly, by counting the number of molecules representing each of the different patterns of phosphorylation, one can obtain a relative quantification of the different phosphorylated proteoforms on the array, and by extrapolation, in the sample from which the array was created.

While described in terms of phosphorylation, it will also be appreciated that a proteoform of a particular protein may represent more than just a single type of modification, e.g., phosphorylation at one or more sequence locations, but may also include additional different types of modifications, e.g., ubiquitylation, methylation, acetylation, nitration, truncation, or any of the other modifications described elsewhere herein.

As will be appreciated, in many cases the affinity reagents used for proteoform analysis will have a higher specificity for their targets than those used in more general proteome analysis described above, where more promiscuous probes (i.e., probes that bind to shorter epitopes and thus multiple different proteins) are used. In particular, probes that are highly specific for epitopes that include the given proteoform variation, e.g., phosphorylation site, insertion, etc., may generally be used for proteoform characterization. Such probes may have affinity for larger sequence segments and contexts than those used in proteome characterization. For example, rather than a trimer or tetramer epitope, proteoform affinity reagents may target longer sequence segments and/or contexts, e.g., 5, 6, 7, 8, 9, 10, 15, 20 or more amino acid residues in sequence or in spatial proximity in a protein's three-dimensional structure. Although discussed in terms of higher affinity probes for proteoform analysis and more promiscuous (or multi-affinity) probes for generalized proteome analysis, it will be appreciated that in either version, the probes used may inform the other analysis, e.g., in a broadscale proteome analysis which identifies proteins one may glean information that is more specific to a particular proteoform that is present. Likewise, where one is seeking to identify the proteoforms of interest present on an array, one may glean broader information about the presence and quantities of proteins on the array, including the protein of interest.

A similar approach may be used to identify proteoforms that represent different splice isoforms or truncations of different protein molecules as well. In particular, one can iteratively probe the protein of interest (and its altered versions) using affinity reagents that target different regions of the protein that may vary among its different forms, e.g., included or excluded exon coded regions, truncated portions, etc., in order to generate a profile of each of the proteins of interest on the array.

In many cases, analyzing and characterizing the proteoforms of different proteins of interest that are present in a sample may involve combinations of the above processes for different types of modifications (e.g., multiple processing modifications and/or different post translational modifications).

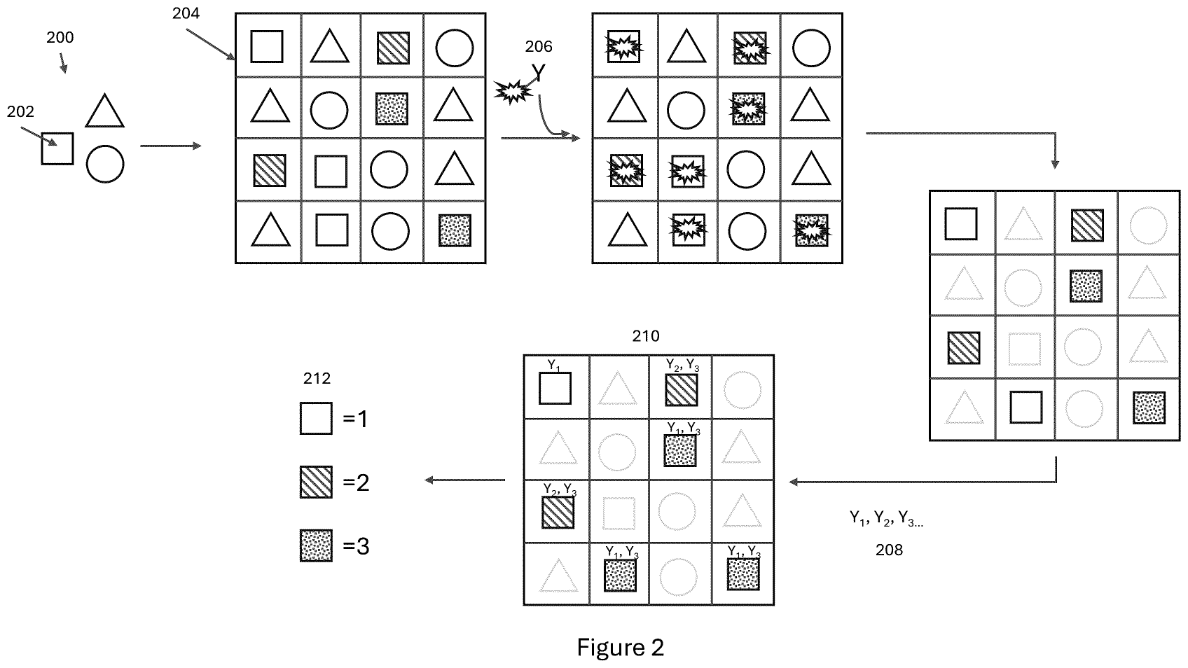

FIG. 2 illustrates a process used for characterizing proteoforms using the methods described herein. As shown, a set of proteins 200, e.g., from a sample, either with or without enrichment or purification, that includes a particular protein of interest 202 (including its various proteoforms and isoforms) is deposited on the surface of an array 204, such that individual protein molecules are separately immobilized and are separately accessible/detectable. As shown, the surface of the array includes a mixture of proteins, including different forms of the protein of interest 202 (shown as 202a, 202b and 202c).

In some cases, the array may be pre-characterized with respect to the location of particular proteins of interest (including their various proteoforms and isoforms), e.g., using the broadscale protein characterization described above. However, while sometimes described in conjunction with the processes for broad scale decoding of larger numbers of proteins on an array, it will be appreciated that in many cases, characterization of all of the proteins on the array other than the protein of interest, may be unnecessary or undesirable. In particular, one may simply wish to identify locations on the array at which the different forms of the protein of interest are located, followed by characterization of which form is present at each such location, without regard for other proteins that are present on the array.

Accordingly, in many cases, and particularly where one is interested in more targeted analysis of specific proteins of interest and their respective proteoforms, the particular proteins of interest may be identified and located using more specific interrogation techniques, e.g., more highly specific affinity reagents that bind very specifically, and thus identify the proteins of interest on the array with relatively high confidence with few or a single interrogation step. This is shown in FIG. 2 where a labeled antibody 206 specific for all forms of the protein of interest 202 is contacted with the array 204. As shown, binding of this antibody provides an indication of the locations on the array occupied by the protein of interest 202 and its various proteoforms and isoforms, e.g., 202a, 202b and 202c.

In some cases, the affinity reagents used to characterize specific proteoforms, and their associated interrogation steps, may provide the locations on the array where all of the different forms of the protein of interest exist, thus obviating the need for a specific step for identifying all possible locations of the protein of interest. In particular, as will be appreciated, in many cases, the higher specificity affinity probes used may allow one to readily identify the locations of the particular protein(s) of interest on the array without the need for broad-scale proteome decoding first. For example, one may interrogate a protein array with affinity reagents specific for one or more species of the particular protein (or proteins) of interest to identify their locations on the array. Interrogations with affinity reagents that are specific for particular modifications would then be used to assign the different modifications to each specific protein location, to provide a characterization of the particular proteoform represented by each protein of interest on the array.

In process, the array 202 that includes the protein of interest in multiple different proteoforms, e.g., proteoform 202a, 202b and 202c, is interrogated using affinity reagents that are specific for different characteristics that make up the different proteoforms. For purposes of illustration, as shown, the protein of interest 202 may include three possible phosphorylation sites within its sequence, and that it may exist as a different proteoform based upon the combination of such sites that are and are not phosphorylated. The resulting proteoforms may include any one of the three sites being phosphorylated, any two of the sites being phosphorylated, all three of the sites being phosphorylated, or none of the sites being phosphorylated. By iteratively interrogating the individual molecules of the protein of interest using affinity reagents specific for phosphorylation at the different positions in the protein of interest, one can easily identify which of eight possible proteoforms is represented at each site using only three affinity reagents, based upon the proteins to which such reagents bind.

By way of illustration, as shown in FIG. 2, the array surface including the protein of interest in its various proteoforms and isoforms, e.g., 202a, 202b and 202c is interrogated with a series of different affinity reagents (Y₁, Y₂ and Y₃ in 208), each specific for a different characteristic of the proteoforms of the protein of interest 202, such as phosphorylation at one of the three phosphorylation sites. By identifying where on the array these antibodies bind (e.g., shown at 210), one can attribute the specific characteristic to the protein located at that position on the array, and thus characterize the particular proteoform or isoform that is located at that position. Because the array represents single molecule localization of proteins, one can then simply count the number of each different proteoform present in order to quantify that proteoform and extrapolate that back to the originating sample (e.g., illustrated at 212).

Although described in terms of probing for a given characteristic of different proteoforms once (e.g., exposing an array to an affinity reagent that targets a given characteristic of a proteoform (or proteoforms) of the protein of interest), in certain preferred cases, a particular characteristic may be probed multiple times to increase the certainty of the identification. In practice, for example, one may re-probe an array that includes proteins of interest in the various proteoforms and isoforms multiple times using the same affinity reagent. Alternatively, one may re-probe the array using multiple affinity reagents that may be different but which recognize and bind to the same characteristic, or which recognize and bind to overlapping sets of characteristics. For example, in some cases, a first probe may bind with high affinity to a protein having two or more specific modifications, while a second may bind with high specificity to a protein having two or more specific modifications, a subset of which overlap (or are the same as) one or more of the modifications of the first protein. By iterative probing, one may elucidate which protein possesses which modifications.

In general, repeated probings or interrogations of an array and the particular proteins of interest with the same affinity reagent, or different affinity reagents with the same or overlapping targets, may be carried out 2, 3, 4, 5, 6, 7, 8, 9, 10 or more times. For example, a particular protein on an array may be probed multiple times using a single type of affinity reagent for a given phosphorylated epitope within that protein. Alternatively, as noted, different affinity reagents that similarly bind to that same phosphorylated epitope may be used to probe the same array of proteins. In certain cases, multiple probings using the same affinity reagent may be performed sequentially, e.g., repeating a particular probing step directly in sequence, e.g., consecutively, following a probing using the same reagent. However, in preferred instances, repeat probings or interrogations with the same reagent (or an affinity reagent targeting the same proteoform characteristic), may be non-sequential, or non-consecutive. For example, where a given analysis requires interrogation of an array of immobilized proteins using four different affinity reagents to different target proteoform characteristics, e.g., affinity reagents A1, A2, A3 and A4, one may separate repeat interrogations with the same reagent by interspersing interrogation with one or more different reagents. As such, an exemplary set of interrogation cycles may be: A1, A2, A3, A4, A1, A2, A3, A4 . . . . Likewise, one may simply intersperse a single reagent interrogation, e.g., A1, A2, A1, A3, A1, A4, etc., or even perform such repeated interrogations in random, albeit non-sequential order. Such multiple probings may increase the confidence in the assessment of binding of an affinity reagent to its expected target epitope.

As will be appreciated, the characteristics of a proteoform may include any of a variety of different types of post translational modifications, splice variations, degradation products, or the like as described above.

Although described for illustration as analysis and characterization of relatively small numbers of proteoforms for any given protein, the number of possible proteoforms for any given protein will generally be dictated by the number of different potential modifications that may be present in a particular protein. Where a protein may potentially include up to n modifications, the number of possible proteoforms of that protein may be upwards of 2ⁿ. Where a particular protein species may contain any number of up to 20 different modifications, that protein could potentially have over 1,000,000 different possible proteoforms.

In the context of the methods described herein, it will be appreciated that one may readily characterize a number of proteoforms for a given protein that is related to the number of detectable modifications for that protein, such that where the number of detectable modifications is equal to y, the number of detectable or characterizable proteoforms for that protein could be up to 2^{y}. A detectable modification will typically include an epitope, the presence or absence of which may be detected, e.g., using the methods described herein, such as epitopes including modified amino acids, truncated or missing epitopes, or the like. In accordance with certain aspects, the methods described herein may use affinity reagents that are specifically able to recognize and bind to such epitopes, allowing one to assess whether they are present or absent in a given protein molecule.

Again, by way of example, where one possesses a library of affinity probes that is capable of characterizing, for example, 12 different modifications to a particular protein species of interest, one would potentially be able to characterize up to 2¹² different potential proteoforms of that protein of interest in a given sample. While the limits of the potential number of proteoforms of any particular protein, or one's ability to detect all possible modifications, may vary, it will be appreciated that for many applications, a predetermined and smaller number of modifications may be deemed more critical for the research at hand. Accordingly, in many cases, one may seek to detect smaller numbers of modifications in a given protein species of interest than are theoretically possible. For example, in many cases, one may simply wish to detect proteoforms that represent patterns of the presence or absence of 1, 2, 3, 4, 5, 6, 7, 8, 9, 10, 11, 12, 13, 14, 15, 16, 17, 18, 19, 20, 30, 40, 50, 60, 70, 80, 90, 100 or more different potential individual modifications to the protein species of interest. As noted above, the number of proteoforms of a given protein of interest increases substantially exponentially with the number of modification sites within that protein (with the caveat that a modification that results in a truncation of a protein of interest may in fact delete residues at which other modifications could occur in the full length protein, and thus potentially reduce the theoretical maximum possible number of modifications), and can readily include anywhere from 2 proteoforms to well over a million proteoforms.

While described above in terms of the possible numbers of modification patterns or proteoforms that could exist in a given protein of interest, given all possible modifications, in biological systems, the number of proteoforms of a given protein of interest may actually be less than the theoretical maximum.

In accordance with the methods, processes, reagents and systems etc. described herein, analysis, characterization, identification and/or quantification of proteoforms of a protein of interest may include individual or separate proteoforms that include all possible modifications to a protein of interest, or it may include proteoform groups that each share a common pattern of a subset of all possible modifications to the protein of interest. In particular, in many cases, one may be desirous of analyzing a subset of modifications in any given protein of interest, e.g., focusing on a pattern of modifications that represents a subset of all possible modifications to the protein of interest that have demonstrated clinical relevance or are otherwise of significant scientific interest. By way of example, for illustration purposes, a given analysis may examine a group of modifications A through E to a given protein of interest, where that protein may have additional possible modifications F through Z. In such cases, identification of a proteoform (or proteoform group) having modifications A through E may include a number of different individual proteoforms that share this same pattern, but differ with respect to potential modifications elsewhere. Thus, for purposes hereof, analysis, detection, and quantification of a given proteoform may relate to such analysis, detection and quantification, etc., of a group of proteoforms that share the common pattern of modifications, while still being heterogeneous with respect to the other modifications, e.g., F through Z.

Relatedly, in some analyses, one may be focused on characterizing a subset of proteoforms in a protein of interest that represents a fraction of the total possible number of proteoforms for that protein, given its different possible biological modifications (e.g., splice forms, PTMs). Similarly, where one utilizes an affinity probe library that is capable of identifying a subset of modifications to a given protein of interest, one may still wish to further focus analysis on a subset of all possible proteoforms that would be characterizable using that set of affinity reagents. In some cases, the subset of possible proteoforms that are characterized may simply relate to those that are actually present within the biological systems, e.g., the particular system just does not create certain modification patterns in any detectable amount. In other cases, certain specific patterns may be identified as being of particular clinical or experimental relevance, e.g., specific proteoforms or proteoform changes being highly correlated and/or causative of specific clinical outcomes. Such proteoforms may reflect significant numbers of modifications (or absence of modifications) in any given protein molecule, but could be focused only on 1, 2, 3, 4, 5, 6, 7, 8, 9, 10, 20, 30, 40, 50, 60, 70, 80, 90, 100 or more such different proteoforms, or focused on less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, despite the potential of much larger numbers of possible proteoforms for that protein. As will be appreciated, the foregoing description specifically includes ranges bounded by the foregoing numbers in relevant combination, e.g., 2 or more patterns and less than 100 patterns, etc.

In some cases, an analysis will only seek to characterize less than 90%, less than 80%, less than 70%, less than 60%, less than 50%, less than 40%, less than 30%, less than 20% or even less than 10% of the total possible number of proteoforms for a set of possible modifications to a protein of interest, whether that set of modifications constitutes all possible modifications or just all possible detectable modifications given the affinity reagent panel used.

With respect to the methods described elsewhere herein, focusing an analysis in accordance with the foregoing may include providing only affinity reagents that are capable of characterizing the reduced number of proteoforms, e.g., foregoing detection of certain irrelevant modifications. Alternatively or additionally, such reduced analyses may utilize bioanalytic processes in decoding the detected proteoforms that ignore less relevant or biologically absent proteoforms.

By way of example, in some cases, one may be focused on the relative abundance of a single particular proteoform or set of proteoforms, e.g., a triple phosphorylated species of a given protein, that may or may not also include other modifications, splice or truncations, etc. vs. any other proteoform of that same species. Alternatively, or additionally, one may be focused on characterizing the relative abundance of a particular proteoform or set of proteoforms with that of potential precursor species, e.g., proteoforms showing double or single phosphorylated species. In other cases, one may look to characterize the relative abundance of hyperphosphorylated species, e.g., triple or quadruple phosphorylated species of the proteins of interest, as indicators of disease onset, progression or severity.

In some cases, a protein-containing sample may be processed to isolate the individual protein molecules contained in that sample, e.g., on the surface of an array as described above. In one part of the process, e.g., an initial step in the process, the particular protein molecule at each location on the array may be identified using a whole proteome analysis technique, such as Prism, as described above. Once the proteins are identified at each location, one can then interrogate the proteins for the different proteoforms using probes that are specific for different proteoforms of the proteins of interest. For example, a protein-containing sample may be analyzed to identify the full range of proteins in that sample, including certain specific proteins of interest that are known to exist in biologically relevant proteoforms. One can then further analyze those specific proteins in those locations on the array to identify and potentially quantify the different proteoforms of that protein on that array, quantifying both the different proteoforms as a fraction of the amount of the protein of interest and as a fraction of the overall proteome present in the sample.

In other cases, a protein of interest may not be present in a sample at levels that are easily analyzed, e.g., they may be below levels where one can assure a representative isolation of such proteins on an array/flow-cell. In these instances, it can be advantageous to enrich for the proteins of interest prior to depositing them onto the array, in order to subsequently analyze the different proteoforms present within the population of such proteins' molecules in the sample. Enrichment can be accomplished using a number of conventional means, including chromatographic enrichment or purification, using any of size exclusion, charge-based separation, relative hydrophobicity, or even using affinity chromatography, to separate and enrich for the protein of interest. In some cases, immune-precipitation techniques, where antibodies to the protein of interest are coupled to beads or other particles, may be used to selectively pull the protein of interest out of solution. The beads are then washed and the protein of interest is then eluted from the beads into a separate fluid, typically at a higher concentration and/or purity than the sample from which it was obtained. As will be appreciated, it will generally be desirable to ensure that any enrichment step, e.g., immunoprecipitation, enriches for a representative pool of the proteoforms present in the sample. For example, in some cases, one may use an antibody in an immunoprecipitation technique that binds specifically to a portion of the protein of interest that is present in all of the proteoforms or isoforms of the particular protein of interest, and/or where that binding is not interfered by the present of one or more proteoform modifications to that protein. Likewise, for other enrichment techniques, e.g., chromatographic purification, one may wish to adopt an enrichment process that enriches for the full proteoform cohort of the protein of interest, including any and all modifications, truncations, insertions etc. As such, where a given proteoform includes a wide range of different splice forms, truncations, etc., a size exclusion-based purification process may not be ideal, as it will separate the differently sized versions of the protein of interest.

In some cases, e.g., where the protein of interest exists in insoluble or less soluble forms, such as may be the case for proteins that may exist in insoluble tangles or plaques in tissue samples, such as Tau and alpha-syn proteins, it may be desirable to solubilize the proteins of interest prior to enrichment. Solubilization may depend upon the nature of the protein of interest, and may include, for example, sarkosyl extraction of insoluble proteins from tissue samples in radio-immunoprecipitation buffer (RIPA) (see, e.g., Singh, et al., Methods Mol. Biol. 2024:2761:317-328).

By way of example, in some cases, immuno-enrichment may involve the use of multiple different antibodies that target and bind to different portions of the protein of interest. This is particularly the case where the protein of interest may exist in multiple different isoforms that may include or lack different portions of the full-length protein, e.g., as a result of splicing variations, post translational processing or degradation, or the like. By using antibodies that target the different regions reflected in those different isoforms, one can target and isolate a larger fraction of all of those isoforms and modified proteins. These antibodies may be used as a pool or in tandem during immunoprecipitation. In the case of immunoprecipitation using bead bound antibodies, these antibodies may again be immobilized on the beads separately where the beads are pooled prior to use in the immunoprecipitation step, or they may be pooled prior to immobilization on the beads.

In some cases, enrichment of the protein of interest may employ a bead-based immunoprecipitation technique where antibodies or antibody binding fragments that are capable of specifically binding to the protein of interest are coupled to solid supports or beads using conventional techniques. These beads are then suspended in a liquid sample containing the protein of interest which are then bound by the antibodies attached to the beads. The beads are then washed to remove any unbound proteins or other materials. The effectiveness of these beads in capturing protein of interest from a mixture can be monitored by a semi-quantitative Western Blot, where a serial dilution of recombinant protein is used as a standard, and the signals from samples before and after immune precipitation can be compared. The specificity of the immunoprecipitation can be examined by using negative controls such as naïve mouse IgG, which would not be expected to cause depletion of the protein of interest. Effectiveness can also be examined by gel staining of the proteins that are enriched by the beads using well established methods like SDS-PAGE followed by Coomassie and silver staining.

Following binding to the beads, the beads may then be subjected to a changed environment in which the binding is weakened. For example, in some cases, the beads may be then exposed to a competitive binder for the antibodies, such as a polypeptide that mimics or duplicates the binding domain or epitope of the protein of interest so as to competitively elute the proteins of interest from the beads. While other conditions may also be employed for elution, including for example, changes in salt concentration, pH etc., this type of elution allows for a more focused elution for the protein of interest as opposed to more stringent conditions that tend to remove a wider variety of specific and non-specifically bound materials form the beads. A variety of different competitive binders may be employed in the context of this type of elution, including poly or oligopeptides, peptide mimics, or other specific binding inhibitors for the antibodies used in the enrichment process. In some preferred cases, these competitive binders may include synthetic peptides designed to mimic or duplicate the sequence of the target epitope of each antibody that was used in the enrichment process, which peptides may be used in molar excess to the antibody. Where, as noted previously, multiple different antibodies having different target epitopes are used in ensuring full enrichment of the protein of interest (and its various proteoforms and isoforms), likewise, multiple mimetic peptides or other competitive binding reagents may be included in the elution process.

Depending on the purity of the bound material and the requirements of the analysis, other nonspecific binding inhibitors, commonly used in disrupting protein-protein interactions, may also be used in the elution of protein of interest from the beads, such as ionic detergents, low pH puffers, chaotropic salts and other denaturants, etc.

In optional cases, additional sample preparation steps may be carried out on the proteins of interest while they are bound to the beads, in order to utilize the advantages of support bound proteins of interest, e.g., in subsequent purification and/or separation steps. For example, in some cases, following binding of the protein of interest to the beads, the bound proteins may be exposed and coupled to the nanoparticles, e.g., SNAPs, used to deposit the proteins of interest in different locations on an array surface. By performing this step on bead bound proteins of interest, one can more effectively remove free particles, i.e., particles that have no associated proteins of interest, through a simple washing step versus a subsequent more complex separation process, e.g., chromatography, filtration, etc.

By employing a single process step as outlined above for both immuno-isolating a protein of interest and coupling such protein to its SNAP, one can analyze far smaller concentrations of a protein of interest in a sample than would be attainable using a multiple step process where losses at each step rapidly deplete the measurable amount of protein of interest in the analyzed sample. For example, in some cases, where a protein is first enriched using a bead-based immunoprecipitation process, where bead bound proteins are eluted and then coupled to SNAPs, it can result in sizable losses at each stage. In one exemplary process, volume requirements, as well as the need for excess proteins and particles needed to drive the proper coupling reactions necessitated a significantly larger starting sample input than would be ideal. For example, where a protein of interest makes up about 0.3% of the mass of a particular type of sample tissue, input sample size may need to be in excess of 400 μg of starting tissue lysate (sample input), to yield a final quantity of protein of interest to analyze using the methods described herein, e.g., in the femtomole or sub-femtomole range. Relatedly, where concentrations are even less, sample inputs become increasingly untenable, e.g., due to lack of sufficient tissue, etc. However, using the single step processes described above, one can use sample inputs of the protein of interest that are far lower, and are at or below 1 ug, 500 μg, 250 pg, 100 μg, 50 pg, 10 μg, 5 pg, 2 μg, 1 pg, 500 ng, 400 ng, 300 ng, 200 ng, 100 ng, 50 ng, 40 ng, 30 ng, 20 ng, 10 ng or even lower, as well as amounts between any two of the foregoing quantities. In any event, measurable amounts of a protein of interest in a sample input may be between 1 ng and 1 ug, between 5 ng and 100 pg, between 5 ng and 5 pg, between 5 ng and 500 ng, between 5 ng and 50 ng, between 10 ng and 1 pg, between 10 ng and 100 ng, and between 10 ng and 50 ng of protein of interest in the starting sample input.

As will be appreciated, when subject to an enrichment step, it may be more difficult to quantify the amount of different proteoforms present in the original sample as a result of the concentration that occurs during the enrichment step for the protein of interest. Accordingly, in some cases, standards may be included in the sample, prior to the enrichment step to provide a basis for tracking how much protein was present originally and how that was impacted by the enrichment step. For example, in some cases, a known amount of the protein of interest, that is separately identifiable form the endogenous protein of interest, may be spiked into the sample. By tracking the standard or control protein through the process and quantifying what was detected at the back-end, one can extrapolate a similar partitioning of the endogenous protein of interest, and thus get a relative quantitation of such protein in the original sample. Providing the protein of interest as a separately identifiable control can be a matter of adding a detectable label or tag to such standard protein in order to later identify it during the analysis process. Such tags may include chemical tags that may be modified to be detected, fluorescent tags that may be detected using fluorescence microscopy, or biochemical tags that may be recognized by specific probe moieties, e.g., antibodies, or other highly specific binding groups, such as biotin or streptavidin, such that the standard version of the protein may be identified and distinguished from the protein of interest that originates from the sample. Alternatively, rather than adding standard proteins to the sample material, one may also optionally run parallel analyses using standard “samples” where the amount of the protein of interest is known. Based upon the yield of the standard process, one can make an assumption that the true sample was processed with similar yields. As will be appreciated, one could potentially run multiple “standard samples” that included different amounts of the protein of interest in order to create a quantity curve in order to even better assess the abundance of the protein of interest in the true sample.

### B. Proteoform Pattern Characterization and Monitoring

The proteoform characterization methods and systems described herein are particularly useful in characterizing broader patterns of proteoforms present in a given sample or across multiple samples. In particular, for many proteins, there exist numerous potential modifications at numerous different sites within the protein or of numerous different types, including e.g., splice variations. As these may exist alone or in any number of combinations, a number of different proteoforms may exist in a given sample for a given species of protein at any given time. In some cases, the different patterns of proteoforms in a sample (presence, quantity, relative abundance, etc.) may have different and important implications related to the function of biological system from which the sample was derived.

The functionality of the methods, processes, systems and reagents described herein provides an apt analogy of the complexity of proteoforms in biological systems. In particular, the methods, processes, systems and reagents described herein are capable of characterizing multiple levels of exponentiation of biological complexity related to proteins and proteoforms that have previously been unmeasurable.

For example, at a first level of complexity, and as described in detail herein, one may readily detect modifications at numerous sites within a molecule of a particular protein of interest from a sample to derive a pattern of modifications (or a proteoform) within that protein. In a further level of complexity, one may ascertain multiple patterns of modifications (or multiple proteoforms) across multiple molecules of the particular protein of interest from a sample. In still another level of complexity, using the platform described herein, one may readily quantify each of those proteoforms in a sample to provide relative abundances of each. As an added complexity, one may further ascertain and compare the proteoforms present and their relative abundances across multiple samples, to compare shifts in those patterns and/or their relative abundances between different samples, e.g., healthy vs. diseased, a given patient's samples from different times, samples pre and post treatment or intervention (or hypothesized intervention), etc. Lastly, given the broad sensitivity of the platform described herein, one could do all of the foregoing with multiple different proteins of interest.

As described above, for any given protein of interest, the methods described herein are readily able to characterize the presence or absence of 1, 2, 3, 4, 5, 6, 7, 8, 9, 10, 11, 12, 13, 14, 15, 16, 17, 18, 19, 20, 30, 40, 50, 60, 70, 80, 90, 100 or more different modifications within a protein of interest. The detected modifications within a given molecule of a protein of interest make up a pattern of modifications to that protein, or a proteoform, that is present in the sample analyzed. By detecting these modifications across multiple molecules of the protein of interest in the sample, one can characterize multiple patterns of modifications or proteoforms of the protein of interest that are present in the sample. In particular, as noted previously, for a given protein of interest, one may readily characterize from 1 to millions of different proteoforms of a protein of interest, but in preferred cases, may characterize 1, 2, 3, 4, 5, 6, 7, 8, 9, 10, 20, 30, 40, 50, 60, 70, 80, 90, 100 or more differing proteoforms of the protein of interest, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

In some cases, the mere presence or absence of different proteoforms in a sample, or over time in a biological system, may provide only one aspect of important information. In many cases, a biological system may maintain some, many or all of the same proteoforms during periods of biological change, but the ratios of the abundances of different proteoforms present at any given time may change and be reflective of biological change. For example, a healthy patient's sample may reflect a pattern of proteoforms for a given protein of interest, where the different proteoforms are present in the sample at a first set of abundance ratios, whereas the same proteoforms present in a diseased patient's samples may be present at measurably different abundance ratios, indicating the diseased state. Moreover, by monitoring a patient over time and examining these ratios, one may be able to identify inflection points in the potential onset of disease in otherwise healthy patients. For example, by characterizing and quantifying the various different proteoforms of one or more proteins of interest in a sample, one can develop a pattern or set of ratios of proteoform abundances in that sample, and compare that pattern of proteoform abundances to other samples, e.g., healthy vs. diseased patient samples, monitored patients over time, treated and untreated samples, e.g., for identifying candidates for disease prevention or intervention, etc.

Accordingly, in addition to being able to characterize which proteoforms of a given molecule of interest are present in a sample, using the methods, processes, systems and reagents described herein, one can also quantify the amounts or relative abundances of each proteoform present in a given sample. In addition, because the methods described herein characterize the proteoforms on a single molecule basis, one can potentially quantify the number of protein molecules that represent each of the various proteoforms of the protein of interest are present at extremely high dynamic range, e.g., measuring abundances of different proteoforms over 9 orders of magnitude, or from, e.g., 1 molecule to billions of molecules or even greater. By way of example, one may measure different proteoforms within a sample where the relative abundances between any two proteoforms in the sample may differ by less than 1 order of magnitude, more than 1 order of magnitude, more than 2 orders of magnitude, more than 3 orders of magnitude, more than 4 orders of magnitude, more than 5 orders of magnitude, more than 6 orders of magnitude, more than 7 orders of magnitude, more than 8 orders of magnitude, or more than 9 orders of magnitude.

As will be appreciated, the significant detection dynamic range of the methods and processes described herein provides significant advantages in detecting and quantifying rare proteoforms of any given protein of interest among populations of potentially hundreds, thousands, 10s of thousands, 100s or thousands, millions, or even billions of other proteins, including other proteoforms of the proteins of interest.

Based upon the relative abundances of the different proteoforms present in a sample, one may provide a proteoform abundance profile of the sample that includes characterization of a plurality of different proteoforms present in that sample (as described above), and the relative abundances of each such proteoform (as also described above). From these proteoform abundance profiles, one may make comparisons among different samples to ascertain changes in biological functions, conditions, etc. impacting those samples. Accordingly, one may compare the proteoform abundance profiles of 2, 3, 4, 5, 6, 7, 8, 9, 10, 20, 30, 40, 50, 60, 70, 80, 90, 100, 200, 300, 500, 1000, 10,000, 100,000, 1,000,000, 5,000,000, 10,000,000, 100,000,000 or more different samples, where such samples may be derived from individual sources or patients, may reflect multiple different sources or patients, may reflect different time courses, different experimental variables, different treatments, or different interventions in biological systems and/or may be derived from biological organisms, model cellular or in vitro systems, or any other source of biological material relevant to the analysis being performed.

In analyzing, characterizing and comparing proteoforms, including proteoform abundance profiles, from multiple samples, certain patterns may emerge as being particularly relevant in the transition of the biological system from which they are derived. For example, the emergence of a particular pattern (appearing or disappearing proteoforms, shifts in proteoform abundance profiles, etc.) may signal the onset of disease or transitioning of a disease state in a patient. The pattern may reflect a particular order of modifications that occur in order for that transition to take place, e.g., a modification at a specific residue that precedes modification at a second specific residue, an increase in a particular proteoform abundance that precedes an increase in another, etc. that signals transition from one state to another. In such cases, comparison of patterns may look to characterize whether such patterns occur as a means of diagnosis, or as a means of measuring whether and to what extent a biological system has transitioned to its subsequent state, e.g., diseased, state. As such, if one is looking for potential effectors of that transition, one may compare samples that are expected to reach that transition state both in the presence and absence of such potential effectors of that transition. In some cases, for example, pharmaceutical candidates or other interventions may be the effector in question. By comparing a system treated with such an intervention and comparing to an untreated sample, where both are reaching a transition point, one can potentially identify drug candidates that have the ability to stop or slow that transition, and potentially prevent the onset, or further progression of that transition, e.g., the disease state.

In addition to the above-described complexity that is readily analyzable using the methods described herein, one may also readily analyze a plurality of different proteins of interest (e.g., as described in greater detail below) from any given sample, simply by using affinity reagents specific for that protein of interest and its modifications. In particular, a given analysis may be able to carry out the characterization of multiple proteoforms and their relative abundances on one or more samples as described above, but on 2, 3, 4, 5, 6, 7, 8, 9, 10, 20, 50 or more different specific proteins of interest from each sample.

The exponentiation of both the complexity of biological systems, as well as the power of the methods described herein, may be exemplified with reference to the analysis of a given protein of interest. While in some cases, specific post translational modifications to a protein of interest may have been identified as being relevant to a particular pathology, to date, available tools have been unable to meaningfully characterize and quantify potentially widely varying and different proteoforms that are present in samples sufficient to allow more accurate characterization of their potential roles in the particular disease or biological pathways. Because the methods described herein utilize a single molecule detection method, one can readily characterize large numbers of proteoforms of proteins, including different proteoforms present in clinical samples from patients afflicted with a given disease, the relative abundances of those proteoforms, and comparisons of those, abundance profiles among and between multiple different samples.

For example, proteoforms present in a sample may differ significantly, e.g., in patterns of phosphorylation or relative abundances of one or more different proteoforms, depending upon whether the sample is derived from a healthy patient, a diseased patient or a patient with more aggressive forms of a disease.

Accordingly, with respect to the analysis and characterization of the proteins of interest in samples, using the methods, processes, systems, reagents and components thereof (referred collectively herein as “platform” for ease of reference) described herein, one could potentially detect any number of the potential modifications to those proteins, e.g., as set forth in Tables 1 through 7, below. Further, one may readily characterize the relative abundances of the different proteoforms present in any sample, and then compare those among multiple samples to identify potential progression pathways, potential interventions, or potential diagnostic indicators of disease onset or progression.

From a general perspective, and in a simple sense, one may characterize a state of progression of disease in a patient by determining the relative abundance of two or more proteoforms, such as the ratio of protein that is phosphorylated at a first location and protein that is phosphorylated at the first location and a second location, from samples that reflect different time points for a patient who is suffering, or potentially suffering from a disease. That relative abundance or ratio may be indicative of the progression of a given disease.

### III. Proteoform Analysis of Significant Biological Proteins of Interest

There are a number of diseases, and biological conditions, for which the biological causes, triggers, and indicators have proved elusive to scientists and the healthcare field. This is particularly true in the fields of oncology, neurobiology and cardiology, where the complexity of biological functions potentially involved in diseases or conditions makes pinpointing specific pathology causes, predictive indicators, and targetable biological pathways very difficult. This is not simply due to the number of protein pathways involved in these systems, but also to the sheer number of different versions, or proteoforms, of each involved protein that may exist and contribute functionally to those biological processes and systems.

### A. Catenin Beta-1

By way of example, a number of proteins have been identified as being implicated in pathways associated with cancer onset, progression, severity and treatability.

One such protein of interest is Catenin beta-1, the product of the CTNNB1 gene, also referred to as β-catenin. Catenin beta-1 is a dual function protein involved in regulation and coordination of cell-cell adhesion and gene transcription, and mutations and overexpression of catenin beta have been associated with many cancers, including hepatocellular carcinoma, colorectal carcinoma, lung cancer, malignant breast cancer, ovarian and endometrial cancers. Moreover, and of particular relevance here, is that regulation and degradation of catenin beta-1 protein has been shown to be controlled by ubiquitylation and phosphorylation at a number of sites in its amino acid sequence (see, e.g., Tominaga et al., Genes Cells (2008) 13(1):67-77. doi: 10.11l/j.1365-2443.2007.01149Shah et al. Front Oncol. 2022 Mar. 14; 122:858782. Doi:103389/fonc.202285878). Accordingly, it is of significant advantage to be able to characterize the various proteoforms of catenin beta-1 in biological samples to better elucidate the role that different proteoforms of the protein may play in various cancer-related pathways.

Given the role of post translational modifications in regulation of the expression and presence of the catenin beta protein that is implicated in multiple cancer pathologies, it is desirable to be able to ascertain the patterns of modifications present in biological samples as a function of whether and where a sample sits in a particular pathology, its response to external influences, e.g., drug or drug candidates, potential causative agents etc. As will be appreciated, the methods, processes, systems, and reagents described herein are particularly useful in identifying modifications to individual catenin beta protein molecules, mapping patterns of modifications (or proteoforms) within those individual protein molecules, quantifying those proteoforms within biological samples, and comparing those quantified catenin beta-1 proteoforms across samples to achieve the above-noted objectives and more.

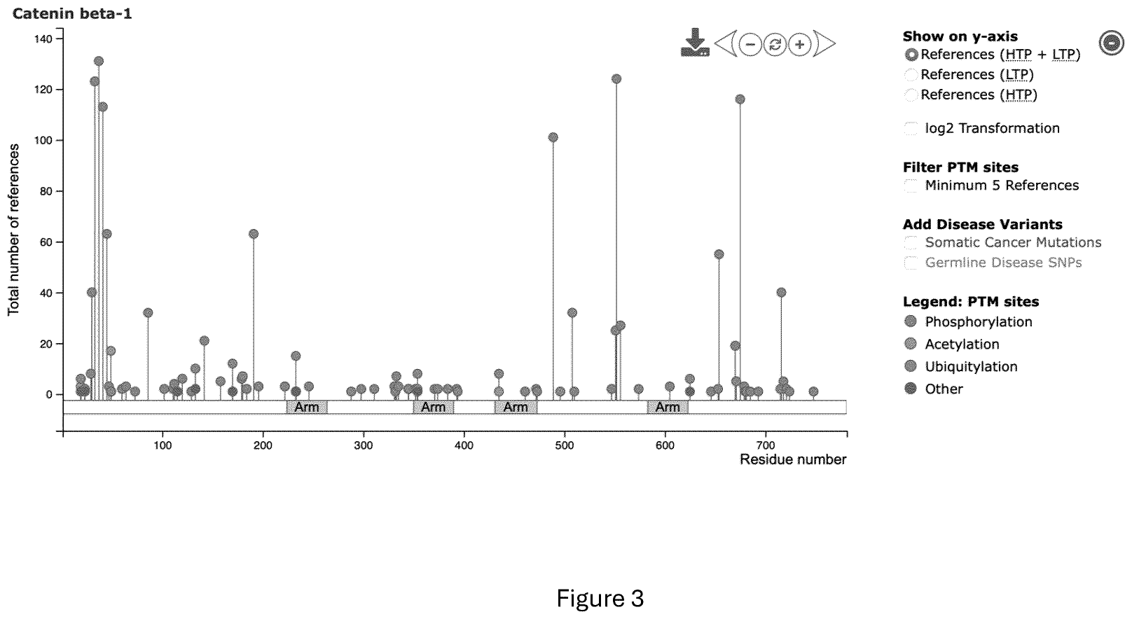

Table 1, below, and FIG. 3 provide detailed listings and a schematic of a number of different identified post-translational modifications (site and type) for catenin beta-1 (see phosphosite.org). For ease of illustration, the tables below provide listings of amino acid residues and their locations in the full-length proteins, along with a notation of the modification.

TABLE 1
Catenin Beta-1 Modification

| Acet. | Ubiq. | Phos. | | | | Meth. | Csp | Nedd. |
|---|---|---|---|---|---|---|---|---|
| acK49 | ubK49 | pY30 | pY142 | pT384 | pY654 | meK49 | caD115 | neK170 |
| acK345 | ubK133 | pS33 | pS179 | pT393 | pY670 | m3K49 | | neK233 |
| acK354 | ubK158 | pS37 | pS184 | pT461 | pS675 | m1K133 | | neK354 |
| acK435 | ubK170 | pT41 | pS191 | pT472 | pT679 | | | neK625 |
| | ubK180 | pS45 | pS196 | pS473 | pS680 | | | |
| | ubK233 | pS47 | pS222 | pY489 | pS681 | | | |
| | ubK288 | pS60 | pS246 | pT510 | pT685 | | | |
| | ubK335 | pY64 | pT298 | pT547 | pT693 | | | |
| | ubK345 | pS73 | pS311 | pT551 | pS715 | | | |
| | ubK354 | pY86 | pY331 | pS552 | pY716 | | | |
| | ubK394 | pT102 | pT332 | pT556 | pS718 | | | |
| | ubK435 | pS111 | pY333 | pT574 | pS721 | | | |
| | ubK496 | pT112 | pS352 | pS605 | pY724 | | | |
| | ubK508 | pT120 | pT371 | pS646 | pY748 | | | |
| | ubK625 | pS129 | pS374 | pT653 | | | | |
| | ubK671 | | | | | | | |

Acet. = acetylation,
Ubiq = ubiquitylation,
Meth = methylation,
CSP = Caspase cleavage site,
nedd = neddylation,
m1 = monomethylation,
m2 = dimethylation and
m3 = trimethylation

It will be appreciated that the methods, processes, systems, arrays and reagents described above generally for proteins of interest are directly applicable to the analysis of catenin beta 1 proteoforms that include one or more of the modifications described in Table 1, above. For example, the analyses described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different modifications within the catenin beta-1 protein, that may generally include any one or more of the modifications set forth in Tables 1 above. The detected modifications within a given molecule of catenin beta-1 protein make up a pattern of modifications to that protein, or a proteoform, that is present in the sample analyzed. By detecting these modifications across multiple molecules of the catenin beta-1 protein in the sample, one can characterize multiple patterns of modifications or proteoforms of the catenin beta-1 protein that are present in the sample. In particular, as noted previously, one may readily have the ability to characterize from 1 to millions of different proteoforms of a catenin beta-1 protein, but in preferred cases, may characterize 1, 2, 3, 4, 5, 6, 7, 8, 9, 10, 20, 30, 40, 50, 60, 70, 80, 90, 100 or more differing proteoforms of catenin beta-1 protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

While the methods, processes, systems, reagents etc. described herein may be employed to identify most if not all of the above-referenced modifications to the catenin beta protein, and in turn characterize proteoforms that include each of those modifications, in many cases, preferred analyses will focus on one or both of phosphorylation and/or ubiquitylation modifications to the protein, as these have been specifically tied to processes that have been implicated in cancer onset and progression, e.g., catenin beta-1 overexpression and mutation. As such, in preferred aspects, a plurality of the modifications that are analyzed and detected may be phosphorylation modifications and/or ubiquitylation modifications to the catenin beta-1 protein. In some cases, the analysis may detect and identify at least 2, 3, 4, 5, 6, 7, 8, 9, 10, 11, 12, 13, 14, 15, 16, 17, 18, 19, 20 or more phosphorylation modifications set forth in Table 1, above, within the catenin beta-1 protein. Likewise, in some cases, the analysis may detect and identify at least 2, 3, 4, 5, 6, 7, 8, 9, 10, 11, 12, 13, 14, 15, 16, 17, 18, 19, 20 or more ubiquitylation modifications set forth in Table 1, above, within the catenin beta-1 protein.

In some cases, preferred analyses may focus on the phosphorylated serine, phosphorylated threonine and/or phosphorylated tyrosine residues within the protein's sequence as set forth in Table 1, as these have been shown to form binding domains for associated pathway proteins (see, e.g., Shah, supra). Accordingly, in some cases, the analyses described herein will focus identification of the presence or absence of at least 1, 2, 3, 4, 5, 6, 7, 8, 9, 10, 11, 12, 13, 14, 15, 16, 17, 18, 19, 20 or more of the above-referenced phosphoserine, phosphothreonine and/or phosphotyrosine modifications within the catenin beta-1 proteins within the sample.

As described in detail elsewhere herein, where catenin beta-1 is the protein of interest, one may characterize multiple proteoforms of the protein present in a sample, quantify those proteoforms, and compare those quantified proteoforms across multiple different samples, e.g., health vs. diseased tissues, treated vs. untreated patients, etc.

### B. MAPK1/ERK2

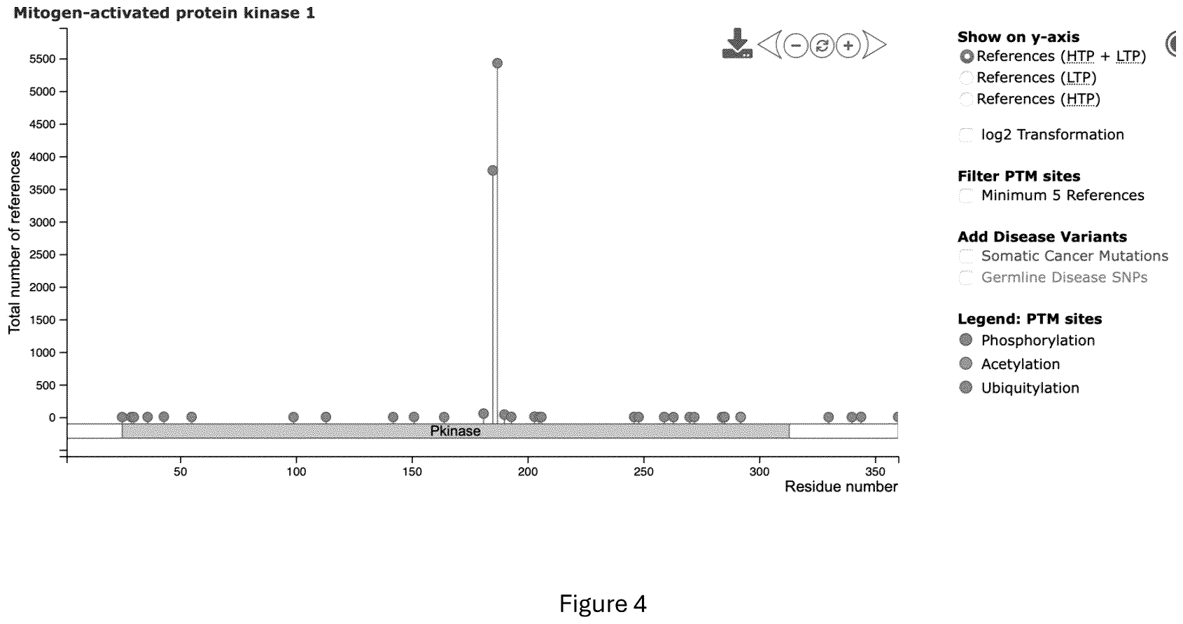

Another protein of interest that has been implicated in cancer-relevant biological pathways is mitogen activated protein kinase 1-ERK2 (also referred to herein as the “ERK2 protein”). The ERK2 kinase is a protein that is widely involved in eukaryotic cell signal transduction that has been implicated in multiple different cancers in either mutated or overexpressed forms. As with catenin beta-1 above, post translational modification, and particularly phosphorylation and ubiquitylation of the ERK2 kinase protein factors into the regulation of the protein levels and activities within biological systems. Accordingly, analysis of those and other modifications and the proteoform patterns and quantities in biological systems is of significant interest to scientific and medical researchers. Table 2, below, and FIG. 4 provide detailed listings and a schematic of a number of different identified post-translational modifications (site and type) for the ERK2 protein.

TABLE 2
ERK2 Modification

| | |
|---|---|
| Ubiq. | Phos. |

| | | | |
|---|---|---|---|
| ubK55 | ubK272 | pY25 | pY187 |
| ubK99 | ubK285 | pS29 | pT190 |
| ubK151 | ubK292 | pY30 | pY193 |
| ubK164 | ubK330 | pY36 | pY205 |
| ubK203 | ubK340 | pY43 | pT206 |
| ubK259 | ubK344 | pY113 | pS246 |
| ubK270 | | pS142 | pS248 |
| | | pT181 | pY263 |
| | | pT185 | pS284 |
| | | pY187 | pS360 |
| | | pT190 | |

Ubiq = ubiquitylation, phos = phosphorylation

As above, it will be appreciated that the methods, processes, systems, arrays and reagents described above generally for proteins of interest are directly applicable to the analysis of ERK2 proteoforms that include one or more of the modifications described in Table 2, above. For example, the analyses described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different phosphorylation and/or ubiquitylation modifications within the ERK2 protein, that may generally include any one or more of the modifications set forth in Tables 2 above. The detected modifications within a given molecule of ERK2 protein make up a pattern of modifications to that protein, or a proteoform, that is present in the sample analyzed. By detecting these modifications across multiple molecules of the ERK2 protein in the sample, one can characterize multiple patterns of modifications or proteoforms of the ERK2 protein that are present in the sample. In particular, as noted previously, one may readily have the ability to characterize from 1 to millions of different proteoforms of a ERK2 protein, but in preferred cases, may characterize 1, 2, 3, 4, 5, 6, 7, 8, 9, 10, 20, 30, 40, 50, 60, 70, 80, 90, 100 or more differing proteoforms of ERK2 protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

As described in detail elsewhere herein, where ERK2 is the protein of interest, one may characterize multiple proteoforms of the protein present in a sample, quantify those proteoforms, and compare those quantified proteoforms across multiple different samples, e.g., healthy vs. diseased tissues, treated vs. untreated patients, etc.

### C. EGFR

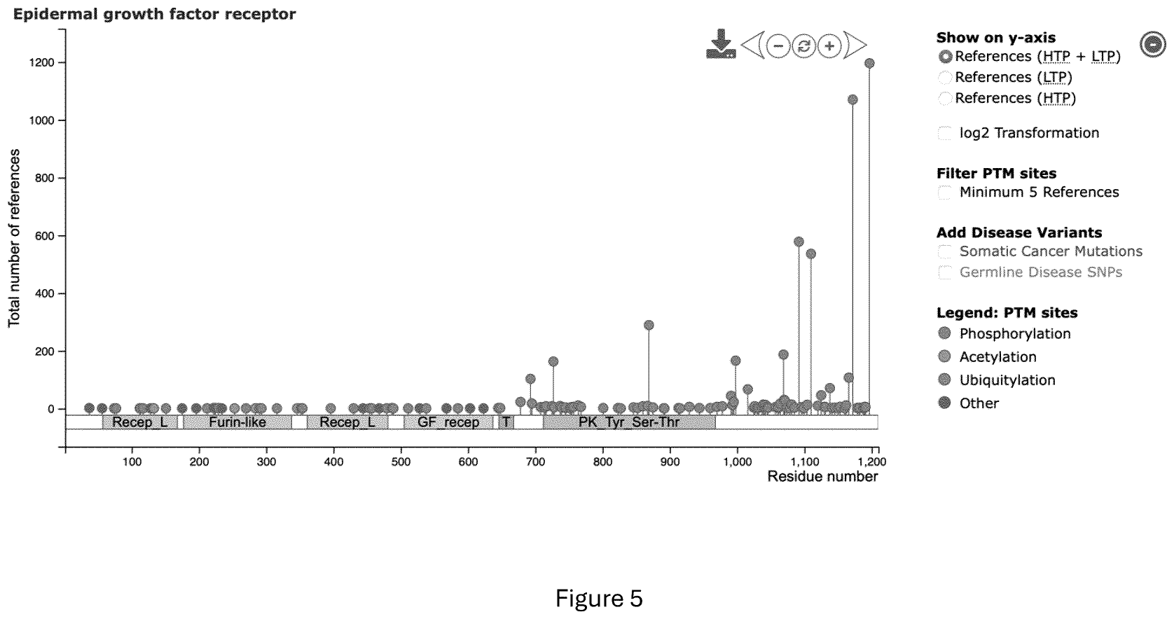

Another significant protein of interest is the epidermal growth factor receptor protein (EGFR). EGFR is a protein from a family of closely related receptor tyrosine kinases whose physiological function lies in regulation of epithelial tissue development and homeostasis. It has been implicated as a driver of tumorigenesis in many cancers, such as glioblastoma, lung and breast cancers (see, e.g., Sigismund et al. Mol Oncol. 2017 Nov. 27; 12(1)3-20). As with the other proteins of interest described herein, modifications to residues of the EGFR protein, like phosphorylation, ubiquitylation, and others can have significant impacts on the function of the protein, and may have implications in its function in cancer biology. As such, characterization, quantification and examination of the different proteoforms of EGFR may be of significant scientific and medical interest as described for the other proteins of interest herein. Table 3, below, and FIG. 5 provide detailed listings and a schematic of a number of different identified post-translational modifications (site and type) for the EGFR protein. (see, e.g., phosphosite.org)

TABLE 3
EGFR Modification

| Acet. | Ubiq. | | Phos. | | | Meth. | Sum. | Glycos. |
|---|---|---|---|---|---|---|---|---|
| acK133 | ubK212 | ubK823 | pY74 | pY869 | pS1071 | m2R222 | smK37 | glN56 |
| acK253 | ubK293 | ubK846 | pS77 | pY891 | pT1074 | m2R224 | | glN128 |
| acK284 | ubK396 | ubK852 | pY112 | pT892 | pT1078 | m1K745 | | glN175 |
| acK346 | ubK454 | ubK860 | pY113 | pY915 | pS1081 | m1K1188 | | glN196 |
| acK1061 | ubK479 | ubK867 | pY117 | pY944 | pT1085 | | | glN234 |
| acK1179 | ubK487 | ubK875 | pS151 | pY978 | pY1092 | | | glN352 |
| acK1182 | ubK489 | ubK913 | pS229 | pS991 | pS1096 | | | glN444 |
| | ubK538 | ubK929 | pY270 | pT993 | pS1104 | | | glN528 |
| | ubK708 | ubK960 | pT290 | pS995 | pY1110 | | | glN568 |
| | ubK713 | ubK970 | pY316 | pY998 | pS1120 | | | glN603 |
| | ubK714 | ubK1061 | pT354 | pY1016 | pY1125 | | | glN623 |
| | ubK716 | ubK1099 | pT430 | pS1025 | pS1130 | | | |
| | ubK728 | ubK1160 | pS457 | pS1026 | pT1131 | | | |
| | ubK737 | ubK1179 | pS511 | pS1030 | pY1138 | | | |
| | ubK739 | ubK1182 | pY585 | pT1032 | pT1141 | | | |
| | ubK754 | ubK1188 | pS645 | pS1037 | pT1145 | | | |
| | ubK757 | | pT648 | pS1039 | pT1150 | | | |
| | | | pT678 | pT1041 | pS1153 | | | |
| | | | pT693 | pS1042 | pS1162 | | | |
| | | | pS695 | pS1045 | pS1166 | | | |
| | | | pT725 | pT1046 | pY1172 | | | |
| | | | pY727 | pS1057 | pS1190 | | | |
| | | | pS752 | pS1064 | pT1191 | | | |
| | | | pY801 | pY1069 | pY1197 | | | |
| | | | pY827 | pS1070 | | | | |

Acet. = acetylation,
Ubiq = ubiquitylation,
Meth = methylation,
m1 = monomethylation,
m2 = dimethylation

It will be appreciated that the methods, processes, systems, arrays and reagents described above generally for proteins of interest are directly applicable to the analysis of EGFR proteoforms that include one or more of the modifications described in Table 3, above. For example, such analyses as described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different phosphorylation and/or ubiquitylation modifications within the EGFR protein, that may generally include any one or more of the modifications set forth in Table 3 above. The detected modifications within a given molecule of EGFR protein make up a pattern of modifications to that protein, or a proteoform, that is present in the sample analyzed. By detecting these modifications across multiple molecules of the EGFR protein in the sample, one can characterize multiple patterns of modifications or proteoforms of the EGFR protein that are present in the sample. In particular, as noted previously, one may readily have the ability to characterize from 1 to millions of different proteoforms of a EGFR protein, but in preferred cases, may characterize 1, 2, 3, 4, 5, 6, 7, 8, 9, 10, 20, 30, 40, 50, 60, 70, 80, 90, 100 or more differing proteoforms of EGFR protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

While the methods, processes, systems, reagents etc. described herein may be employed to identify most if not all of the above-referenced modifications to the EGFR protein, and in turn characterize proteoforms that include each of those modifications, in many cases, preferred analyses will focus on one or both of phosphorylation and/or ubiquitylation modifications to the protein, as these have been cited as more prevalent in cancer pathologies. As such, in preferred aspects, a plurality of the modifications that are analyzed and detected may be phosphorylation modifications and/or ubiquitylation modifications to the EGFR protein. In some cases, the analysis may detect and identify at least 2, 3, 4, 5, 6, 7, 8, 9, 10, 11, 12, 13, 14, 15, 16, 17, 18, 19, 20 or more phosphorylation modifications set forth in Table 1, above, within the EGFR protein. Likewise, in some cases, the analysis may detect and identify at least 2, 3, 4, 5, 6, 7, 8, 9, 10, 11, 12, 13, 14, 15, 16, 17, 18, 19, 20 or more ubiquitylation modifications set forth in Table 3, above, within the EGFR protein.

In some cases, preferred analyses may focus on the phosphorylated serine and/or phosphorylated tyrosine residues, or ubiquitylated lysine residues within the protein's sequence as set forth in Table 3, as these have been shown to form binding domains for associated pathway proteins. Accordingly, in some cases, the analyses described herein will focus identification of the presence or absence of at least 1, 2, 3, 4, 5, 6, 7, 8, 9, 10, 11, 12, 13, 14, 15, 16, 17, 18, 19, 20 or more of the above-referenced phosphoserine, phosphothreonine and/or phosphotyrosine modifications within the EGFR proteins within the sample. Likewise, in some cases, the analyses described herein will focus identification of the presence or absence of at least 1, 2, 3, 4, 5, 6, 7, 8, 9, 10, 11, 12, 13, 14, 15, 16, 17, 18, 19, 20 or more of the above-referenced ubiquitylated lysine residues within the EGFR proteins within the sample.

As described in detail elsewhere herein, where EGFR is the protein of interest, one may characterize multiple proteoforms of the protein present in a sample, quantify those proteoforms, and compare those quantified proteoforms across multiple different samples, e.g., healthy vs. diseased tissues, treated vs. untreated patients, etc.

### D. HER2 (ErbB2)

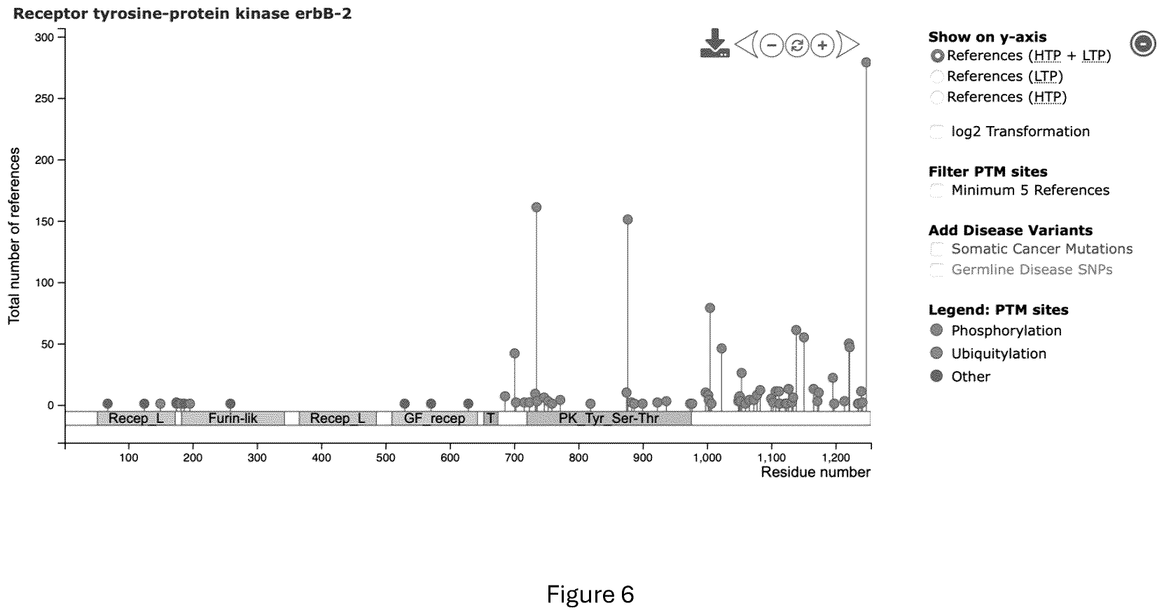

Similar to EGFR, the receptor tyrosine kinase erbB-2, also referred to as HER2 (or human epidermal growth factor receptor 2) is a receptor tyrosine kinase that resides in cellular membranes. As with EGFR above, overexpression of HER2 has been widely implicated in a number of cancers, including breast, stomach, ovarian, and uterine cancers, as well as adenocarcinoma of the lung, and other cancers, and is believed to be regulated through phosphorylation and ubiquitylation, among other processes (see, e.g., Hsu J L, Hung M C (2016) Cancer and Metastasis Reviews. 35(4): 575-588). Table 4, below, and FIG. 6 provide detailed listings and a schematic of a number of different identified post-translational modifications (site and type) for the HER2 protein.

TABLE 4
HER2 Modification

| Ubiq. | Phos. | | | Meth. | Glycos. |
|---|---|---|---|---|---|
| ubK150 | pT182 | pS1007 | pT1132 | m3K175 | glN68 |
| ubK175 | pS196 | pY1023 | pS1134 | | glN125 |
| ubK716 | pT686 | pS1049 | pY1139 | | glN187 |
| ubK724 | pT701 | pS1050 | pS1151 | | N259 |
| ubK736 | pS703 | pS1051 | pT1166 | | N530 |
| ubK747 | pT733 | pT1052 | pT1172 | | N571 |
| ubK753 | pY735 | pS1054 | pT1174 | | N629 |
| ubK883 | pT759 | pT1060 | pY1196 | | |
| ubK887 | pY772 | pS1066 | pT1198 | | |
| ubK937 | pS819 | pS1073 | pS1214 | | |
| | pT875 | pS1078 | pY1221 | | |
| | pY877 | pS1083 | pY1222 | | |
| | pT900 | pS1100 | pS1235 | | |
| | pY923 | pT1103 | pT1236 | | |
| | pS974 | pS1107 | pT1240 | | |
| | pS977 | pY1112 | pT1242 | | |
| | pS998 | pS1113 | pY1248 | | |
| | pS1002 | pS1122 | | | |
| | pT1003 | pT1124 | | | |
| | pY1005 | pY1127 | | | |

As previously described, it will be appreciated that the methods, processes, systems, arrays and reagents described above generally for proteins of interest are directly applicable to the analysis of HER2 proteoforms that include one or more of the modifications described in Table 4, above. For example, as described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different phosphorylation and/or ubiquitylation modifications within the HER2 protein, that may generally include any one or more of the modifications set forth in Table 4 above. As will be appreciated, in many cases, analyses may be focused on the above numbers of modifications that are phosphorylated and/or ubiquitylated residues as set forth in Table 4, above.

The detected modifications within a given molecule of HER2 protein make up a pattern of modifications to that protein, or a proteoform, that is present in the sample analyzed. By detecting these modifications across multiple molecules of the HER2 protein in the sample, one can characterize multiple patterns of modifications or proteoforms of the HER2 protein that are present in the sample. In particular, as noted previously, one may readily have the ability to characterize from 1 to millions of different proteoforms of a HER2 protein, but in preferred cases, may characterize 1, 2, 3, 4, 5, 6, 7, 8, 9, 10, 20, 30, 40, 50, 60, 70, 80, 90, 100 or more differing proteoforms of HER2 protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

As described in detail elsewhere herein, where HER2 is the protein of interest, one may characterize multiple proteoforms of the protein present in a sample, quantify those proteoforms, and compare those quantified proteoforms across multiple different samples, e.g., healthy vs. diseased tissues, treated vs. untreated patients, etc.

### E. Leucine Rich Repeat Serine Threonine-Protein Kinase 2 (LRRK2)

The Leucine rich repeat serine/threonine-protein kinase protein 2 protein (“LRRK2”)has been cited, along with the alpha-synuclein protein, as a key influencer in the onset and progression of neurodegenerative diseases, such as Parkinson's Disease. In particular it has been reported that LRRK2 dysfunction, e.g., through mutation, may influence the accumulation of alpha-synuclein and its pathology to alter cellular functions and signaling pathways by kinase activation of LRRK2 (see, e.g., Rui, et al., Curr Neuropharmacol. 2018 November; 16(9):1348-1357). In many cases, kinase activation, regulation and clearance is driven by post translational modifications, primarily in phosphorylation and/or ubiquitylation, among other modifications. As such, understanding the spectrum of proteoforms of kinases like LRRK2 is of significant interest in understanding the pathways associated with Parkinson's disease.

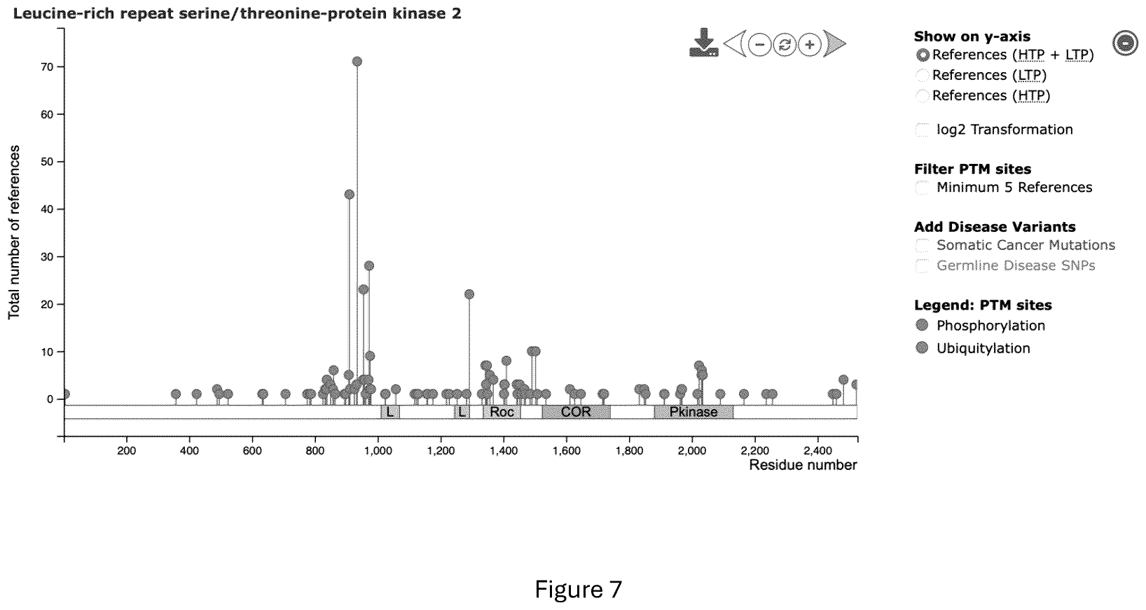

Table 5, below, and FIG. 7 provide detailed listings and a schematic of a number of different identified post-translational modifications (site and type) for the LRRK2 protein.

TABLE 5
LRRK2 Modification

| | |
|---|---|
| Ubiq. | Phos. |

| | | | | |
|---|---|---|---|---|
| ubK1118 | pS3 | pS912 | pT1343 | pY1718 |
| ubK1129 | pS5 | pS926 | pS1345 | pS1721 |
| ubK1833 | pT358 | pS933 | pT1348 | pT1849 |
| ubK1963 | pT424 | pS935 | pT1349 | pS1853 |
| ubK2091 | pT489 | pS954 | pT1357 | pT1912 |
| | pT496 | pS955 | pT1368 | pS1913 |
| | pT524 | pS958 | pY1402 | pT1967 |
| | pS633 | pS962 | pS1403 | pT1969 |
| | pS634 | pS971 | pT1404 | pY2023 |
| | pY636 | pS973 | pT1410 | pT2031 |
| | pY707 | pS975 | pS1443 | pS2032 |
| | pT776 | pS976 | pS1444 | pT2035 |
| | pS784 | pS979 | pS1445 | pS2166 |
| | pS788 | pT1024 | pT1452 | pT2237 |
| | pT826 | pS1025 | pS1457 | pS2257 |
| | pT833 | pS1058 | pS1467 | pY2449 |
| | pS837 | pS1124 | pT1470 | pT2460 |
| | pT838 | pS1157 | pY1485 | pT2483 |
| | pS850 | pS1159 | pT1491 | pT2524 |
| | pS858 | pT1176 | pT1503 | pY2018 |
| | pS860 | pS1219 | pS1508 | |
| | pS865 | pS1228 | pS1536 | |
| | pS895 | pS1253 | pT1612 | |
| | pS898 | pS1283 | pS1627 | |
| | pS908 | pS1292 | pS1647 | |
| | pS910 | pY1332 | pS1716 | |

As previously described, it will be appreciated that the methods, processes, systems, arrays and reagents described above generally for proteins of interest are directly applicable to the analysis of LRRK2 proteoforms that include one or more of the modifications described in Table 5, above. For example, as described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different phosphorylation and/or ubiquitylation modifications within the LRRK2 protein, that may generally include any one or more of the modifications set forth in Table 5 above. As will be appreciated, in many cases, analyses may be focused on the above numbers of modifications that are phosphorylated and/or ubiquitylated residues as set forth in Table 5, above.

The detected modifications within a given molecule of the LRRK2 protein make up a pattern of modifications to that protein, or a proteoform, that is present in the sample analyzed. By detecting these modifications across multiple molecules of the LRRK2 protein in the sample, one can characterize multiple patterns of modifications or proteoforms of the LRRK2 protein that are present in the sample. In particular, as noted previously, one may readily have the ability to characterize from 1 to millions of different proteoforms of a LRRK2 protein, but in preferred cases, may characterize 1, 2, 3, 4, 5, 6, 7, 8, 9, 10, 20, 30, 40, 50, 60, 70, 80, 90, 100 or more differing proteoforms of LRRK2 protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

As described in detail elsewhere herein, where LRRK2 is the protein of interest, one may characterize multiple proteoforms of the protein present in a sample, quantify those proteoforms, and compare those quantified proteoforms across two or more different samples, e.g., healthy vs. diseased tissues, treated vs. untreated patients, etc.

### F. RAC-Alpha Serine Threonine-Protein Kinase (AKT1)

The RAC-alpha serine/threonine protein kinase 1 (“AKT1” and previously known as Protein Kinase B) has been implicated as playing key roles in multiple cell signaling pathways associated with cell metabolism, growth and division, apoptosis suppression and angiogenesis. So it is not surprising that disruptions in the function of this protein have been implicated in a number of cancers, as cell as diabetes, cardiovascular and neurological diseases. As with the other kinases described herein, modification of the protein, and particularly phosphorylation, plays a significant role in activation of the protein and its related pathways, including, for example, phosphorylation at one or both of T308 and S473 (see, e.g., Nitulescu et al. Int J Oncol. 2018 Oct. 16; 53(6):2319-2331). Again, understanding a more comprehensive picture of the various proteoforms of the AKT1 protein and their roles in biological systems, and particularly in cancers and neurodegenerative and other diseases, is of significant clinical and scientific interest. As such, comprehensive analysis of those proteoforms as provided herein can be of significant value.

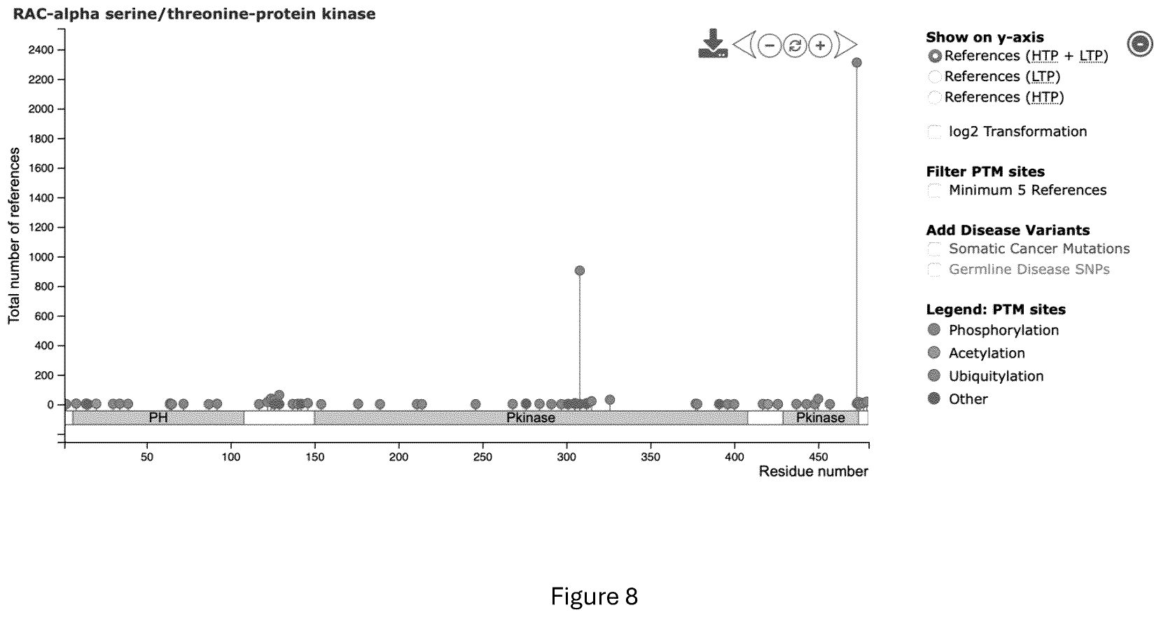

Table 6, below, and FIG. 8 provide detailed listings and a schematic of a number of different identified post-translational modifications (site and type) for the AKT1 protein.

TABLE 6
AKT1 Modification

| Acet. | Ubiq. | Phos. | | Meth. | Sum. | Glycos. |
|---|---|---|---|---|---|---|
| acK14 | ubK8 | pS2 | pT308 | m1K14 | smK64 | glS126 |
| acK20 | ubK14 | pT34 | pT312 | meR15 | smK276 | glS129 |
| acK420 | ubK30 | pT65 | pY315 | meK64 | smK301 | glT305 |
| acK426 | ubK39 | pT72 | pY326 | m3K64 | | glT308 |
| | ubK64 | pT87 | pS378 | m3K140 | | glT312 |
| | ubK140 | pT92 | pS396 | m3K142 | | glS473 |
| | ubK154 | pE117 | pY417 | m2R391 | | |
| | ubK189 | pS122 | pY437 | | | |
| | ubK214 | pS124 | pT443 | | | |
| | ubK268 | pS126 | pT448 | | | |
| | ubK276 | pS129 | pT450 | | | |
| | ubK284 | pS137 | pS457 | | | |
| | ubK297 | pT146 | pS473 | | | |
| | ubK301 | pY176 | pY474 | | | |
| | ubK377 | pT211 | pS475 | | | |
| | ubK400 | pS246 | pS477 | | | |
| | ubK426 | pT291 | pT479 | | | |
| | | pT305 | | | | |

As previously described, it will be appreciated that the methods, processes, systems, arrays and reagents described above generally for proteins of interest are directly applicable to the analysis of AKT1 proteoforms that include one or more of the modifications described in Table 6, above. For example, as described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different phosphorylation and/or ubiquitylation modifications within the AKT1 protein, that may generally include any one or more of the modifications set forth in Table 6 above. As will be appreciated, in many cases, analyses may be focused on the above numbers of modifications that are phosphorylated and/or ubiquitylated residues as set forth in Table 6, above.

The detected modifications within a given molecule of AKT1 protein make up a pattern of modifications to that protein, or a proteoform, that is present in the sample analyzed. By detecting these modifications across multiple molecules of the AKT1 protein in the sample, one can characterize multiple patterns of modifications or proteoforms of the AKT1 protein that are present in the sample. In particular, as noted previously, one may readily have the ability to characterize from 1 to millions of different proteoforms of a AKT1protein, but in preferred cases, may characterize 1, 2, 3, 4, 5, 6, 7, 8, 9, 10, 20, 30, 40, 50, 60, 70, 80, 90, 100 or more differing proteoforms of AKT1 protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

As described in detail elsewhere herein, where AKT1 is the protein of interest, one may characterize multiple proteoforms of the protein present in a sample, quantify those proteoforms, and compare those quantified proteoforms across multiple different samples, e.g., healthy vs. diseased tissues, treated vs. untreated patients, etc.

### G. Mothers Against Decapentaplegic Homolog 2 (SMAD2)

The Mothers against decapentaplegic homolog 2 protein (SMAD2) is a cell signaling protein that mediates the signal of transforming growth factor beta and thus regulates multiple cellular processes, such as cell proliferation, apoptosis and differentiation, and has been implicated in a number of pathologies, including e.g., cancers. Similar to the other proteins of interest, the regulation of the function and expression of the protein typically involves phosphorylation and dephosphorylation of the protein at different loci within its amino acid sequence. Accordingly, understanding the representation of various modified forms of the SMAD2 protein in different contexts, at different stages of biological and pathological functions and processes, is of keen scientific and clinical interest.

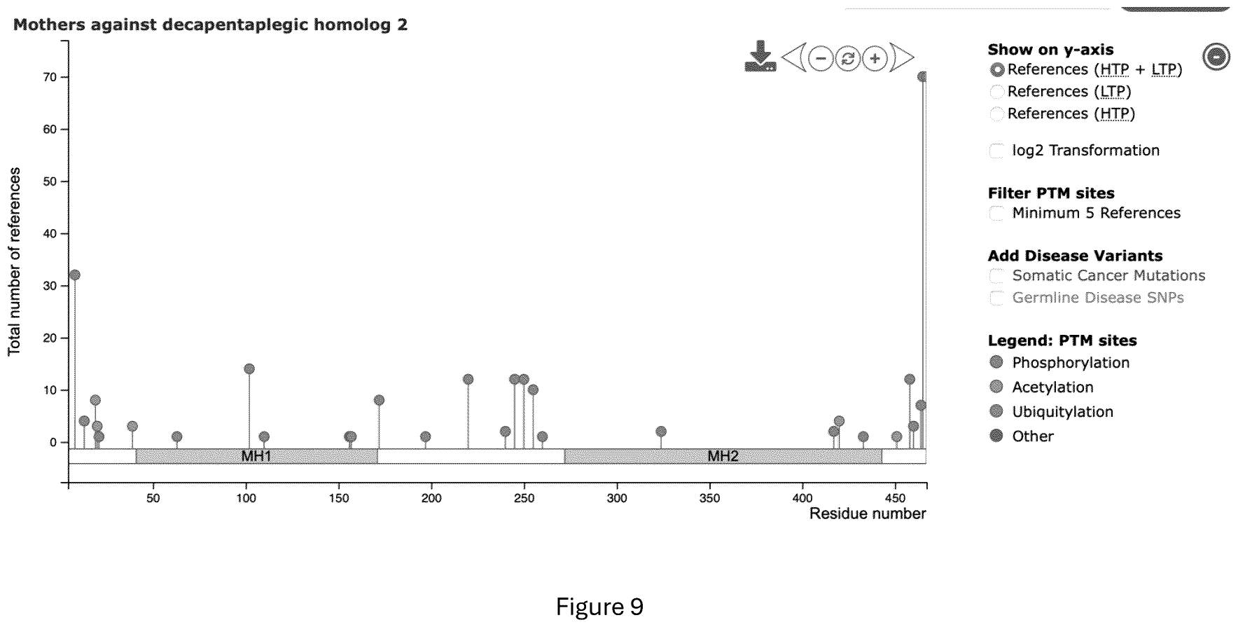

Table 7, below, and FIG. 9 provide detailed listings and a schematic of a number of different identified post-translational modifications (site and type) for the SMAD2 protein.

TABLE 7
SMAD2 Modification

| | | | |
|---|---|---|---|
| Acet. | Ubiq. | Phos. | Sum. |

| | | | | | |
|---|---|---|---|---|---|
| acK19 | ubK13 | pS2 | pT220 | pS417 | smK156 |
| acK20 | ubK63 | pT8 | pS240 | pS433 | |
| acK39 | ubK156 | pS21 | pS245 | pS458 | |
| acK420 | ubK157 | pY102 | pS250 | pS460 | |
| acK451 | | pS110 | pS255 | pS464 | |
| | | pT172 | pS260 | pS465 | |
| | | pT197 | pT324 | pS467 | |

As previously described, it will be appreciated that the methods, processes, systems, arrays and reagents described above generally for proteins of interest are directly applicable to the analysis of SMAD2 proteoforms that include one or more of the modifications described in Table 7, above. For example, as described herein may be focused upon the identification of 1, 2, 3, 4, 5, 6, 7, 8, 9, 10, 11, 12, 13, 14, 15, 16, 17, 18, 19, 20, 30, 40, 50, 60, 70, 80, 90, 100 or more different phosphorylation and/or ubiquitylation modifications within the SMAD2 protein, that may generally include any one or more of the modifications set forth in Table 7 above. As will be appreciated, in many cases, analyses may be focused on the above numbers of modifications that are acetylated, phosphorylated and/or ubiquitylated residues as set forth in Table 7, above.

The detected modifications within a given molecule of SMAD2 protein make up a pattern of modifications to that protein, or a proteoform, that is present in the sample analyzed. By detecting these modifications across multiple molecules of the SMAD2 protein in the sample, one can characterize multiple patterns of modifications or proteoforms of the SMAD2 protein that are present in the sample. In particular, as noted previously, one may readily have the ability to characterize from 1 to millions of different proteoforms of a SMAD2 protein, but in preferred cases, may characterize 1, 2, 3, 4, 5, 6, 7, 8, 9, 10, 20, 30, 40, 50, 60, 70, 80, 90, 100 or more differing proteoforms of SMAD2 protein, or less than 100, less than 90, less than 80, less than 70, less than 60, less than 50, less than 40, less than 30, less than 20, less than 15, less than 10, less than 9, less than 8, less than 7, less than 6, less than 5, less than 4, less than 3, or even only 2 such patterns, with the foregoing description including ranges between any two relevant numbers provided, e.g., 2 or more and less than 100 different proteoforms, etc.

As described in detail elsewhere herein, where SMAD2 is the protein of interest, one may characterize multiple proteoforms of the protein present in a sample, quantify those proteoforms, and compare those quantified proteoforms across multiple different samples, e.g., healthy vs. diseased tissues, treated vs. untreated patients, etc.

As described above, identification and quantitation of the above-described modified forms of the above-described proteins may generally be carried out by the methods described above. For example, a population of proteins may be obtained from a sample that includes as at least a subset, a population of molecules of a given protein of interest that may be heterogeneous with respect to one or more of the various modifications described above, e.g., having one or more proteins that are phosphorylated, ubiquitylated, acetylated, etc., at one or more residues, or representing one or more different truncated or splice forms of the protein. In some cases, such as where the expected concentration of the particular protein of interest may be very low, one may enrich the population of proteins for the various proteoforms that are present relative to other proteins in the sample. Depending upon the abundance of the protein of interest in the sample, one may wish to enrich it relative to total protein in the original sample by 2× or more, 5× or more, 10× or more, 20× or more, 50× or more, 100× or more, or in cases of very low abundance proteins of interest in a sample, 500× or more, 1000× or more, or even 10,000× or more.

As noted above, enrichment may be carried out using affinity enrichment, e.g., via immunoprecipitation, affinity chromatography, size exclusion, ion exchange or other chromatographic techniques. In preferred aspects, an affinity-based enrichment (such as immunoprecipitation or affinity chromatography) is used to enrich for the protein of interest, using antibodies or other affinity reagents that are able to specifically bind across the various modified or truncated forms of the protein of interest.

As will be appreciated, when subject to an enrichment step, it may be more difficult to quantify the amount of different proteoforms present in the original sample as a result of the concentration that occurs during the enrichment step for the protein of interest. Accordingly, in some cases, as described above, standard proteins may be included in the sample, prior to the enrichment step to provide a basis for tracking how much protein was present originally and how that was impacted by the enrichment step. The samples may be enriched for proteins of interest as described above, or where such proteins are sufficiently prevalent in the sample, the samples may be loaded at existing concentrations.

Once the enriched sample is prepared, or in certain cases when no enrichment step is desired, the sample material itself, it may then be coupled to the surface of an array such that individual molecules of the various proteoforms of the particular protein(s) of interest present in the sample are immobilized within discrete locations on the surface of the array, allowing each individual molecule to be individually addressed, e.g., by a detection system. In preferred cases, an individually addressable molecule refers to a molecule that will be able to be observed, e.g. optically, without interference from a neighboring molecule that is immobilized on the array, and includes molecules that may employ associated groups for detection, such as fluorescently labeled affinity reagents as described herein. Array surfaces may include patterned surfaces that allow for localized immobilization, e.g., patterning surface attachment groups that allow binding or immobilization of proteins to those regions. In some cases, the array surfaces may be structured, e.g., including depressions, raised areas or wells or nanowells, in which the proteins may be deposited and immobilized. In some cases, the proteins. May be coupled to particles to facilitate their attachment to the surface, provide for spatial separation from neighboring proteins, and or facilitate their localization and/or substantially single occupancy in desired areas and/or within wells or nanowells.

Array surfaces may provide more than 100, more than 10,000, more than 100,000, more than 1,000,000, more than 10,000,000, more than 100,000,000, or more than 1 billion individual protein or polypeptide molecules disposed on the array surface in individually addressable locations. In some cases, whether or not enriched for a particular protein of interest, an array may reflect more than 10, more than 100, more than 100, or even more than 10,000 different proteins in addition to the protein of interest.

As alluded to above, the methods, processes, systems, devices and reagents described herein may be used to identify, characterize and/or quantify the various proteoforms of the protein(s) of interest that are present in a biological sample. As noted previously, in certain cases, the use of individually addressable molecules of the particular protein of interest (including the various modified forms thereof) may be presented for interrogation using appropriate affinity reagents. In certain cases, such presentation is through immobilization of the individual molecules of the protein(s) of interest on the surface of an array, either in an enriched or non-enriched form, such that individual protein molecules may be individually addressed, e.g., using optical or other detection systems, as described above.

In particular, processes described herein may employ one or more affinity reagents to interrogate those individually addressable molecules to identify the modifications that are represented in those individual molecules. In some cases, the molecules on the array surface may be interrogated by use of detectable affinity reagents, e.g., fluorescently labeled, that specifically bind to different locations on the protein of interest, e.g., in order to identify which proteins may lack certain regions as a result of splice variation or truncation, and/or by affinity reagents that specifically bind to epitopes within the protein that include specific identified modifications, e.g., phosphorylated and/or ubiquitylated amino acid residues. For example, one may employ one or more affinity reagents that specifically bind to an ERK2 or EGFR protein that possesses a given modification at a given amino acid residue as set forth in any of Tables 1 through 7 above. By way of example, for ERK2 affinity may be for epitopes that include, e.g., phosphorylation at any one or more of, e.g., tyrosines 25, 36, 43, 187, 193, 263, etc., at threonines 181, 185, 190, 206, or at serines 29, 142, 246, 248, 284, 360, etc. Similarly, for EGFR affinity may be for epitopes that include, e.g., phosphorylation at any one or more of, e.g., tyrosines 74, 112, 113, 117, 270, 316, 585, 727, 764, 827, 869, etc., threonines 290, 354, 430, 648, 678, 693, 725, 892, 993, 1032, 1041, etc., or serines 151, 229, 457, 511, 645, 695, 752, 768, 991, 995, 1025, 1026, 1030, 1042, 1045, 1057, 1064, etc. As will be appreciated, the foregoing lists of modifications are intended to be exemplary and not to be exhaustive or otherwise limiting. As noted previously, in some cases, an analysis will include detection of subsets of the above-described modifications to the various proteins on the array surface that may include more highly relevant modifications.

In certain cases, an assay may be tailored to use affinity reagents to all or subsets of the above-described modifications. For example, where one is assessing the abundance of a given specific proteoform that includes only a subset of phosphorylation sites described above, one would only need to interrogate the array with a subset of the above-referenced affinity reagents, e.g., to detect the specific modifications, as well as any splice forms or controls or standards.

In preferred aspects, multiple different affinity reagents are used that specifically bind to different modified forms of the protein. For example, one may interrogate the immobilized individual molecules of the protein(s) of interest iteratively using affinity reagents, such as antibodies, antibody fragments, aptamers or mini-binding proteins, that specifically bind to the particular molecules of the protein of interest that possess each of the specific modifications being analyzed, later assembling a tally of which modifications were present in which individual molecules at each location on the array occupied by a molecule of the protein of interest. Depending upon the desired scope of the analysis and characterization, one may iteratively interrogate the array surface with 1, 2, 3, 4, 5, 6, 7, 8, 9, 10, 15, 20, 25, 30, 35, 40, 45, 50, 60, 70, 80, 90, 100 or more different affinity reagents having specificity for different modifications of the protein of interest. Although described as iterative interrogation, it will be understood that in some cases, interrogation may be carried out in parallel, e.g., using differentially labeled and/or separately detectable affinity reagents.

As will be appreciated, in many cases, a single protein may be multiply modified, both as to a given type of modification, e.g., phosphorylation, as well as to different types of modifications, e.g., phosphorylation at one or more residues and ubiquitylation at one or more residues the protein.

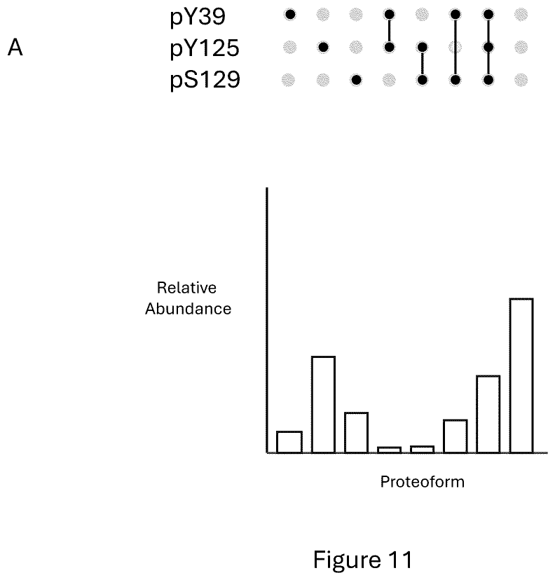

Binding (or non-binding) of these affinity reagents is then detected at each cycle at the various locations on the array at which the protein of interest is located, and the aggregated binding information is used to identify which form of the protein is present at each location (which splice form it is and/or which post translational modifications it includes). Each present modification pattern may then be generally quantified by counting the number of times a given pattern of modifications is reflected across the identified molecules of the protein of interest on the array (See, e.g., FIG. 11).

In addition to the use of affinity reagents specific for different modified epitopes of a protein of interest, in some cases, additional affinity probes may be employed, e.g., that have affinity for different epitope sequences present in the full length protein, e.g., using the approach outlined in, e.g., U.S. Pat. Nos. 10,473,654B1, 11,545,234B1, and Eggertson, et al. bioRxiv, the full disclosures of which were previously incorporated herein by reference in their entirety for all purposes. Briefly, additional use of such multi-affinity probes can provide additional protein sequence information in the identification and characterization of different forms of the protein of interest, e.g., reflecting different binding among different truncations, modifications, etc.

In many cases, an assay performed on an array as set forth above may include suitable controls. For example, in some cases, a “null” lane or portion of an array may be utilized where no proteins of interest are deposited, so that one can ascertain a level of nonspecific binding of affinity reagents to the array surfaces, absent any proteins of interest. Additionally, standard proteins of interest, including standards that bear one or more post translational modifications that are the subject of analysis may be employed, either as spike in controls, or separate controls, e.g., run on their own lane in a flow cell based array (e.g., as described below), in order to assist in quantification and characterization of the actual sample proteins. Different controls may be produced by purification from natural samples, or through synthetic means, e.g., cloning and expression of specific versions of the proteins of interest, and/or targeted or untargeted post translational modification of the standard proteins of interest.

Although described in terms of characterization and quantification of individual proteoforms present in a sample, e.g., proteins bearing a certain modification or set of modifications, it will be appreciated that in many cases, the methods described herein will be used to characterize and quantify sets of different proteoforms that are present in any given sample.

For example, analysis of a single sample may identify the presence and relative abundance of 1, 2, 3, 4, 5, 6, 7, 8, 9, 10, 15, 20, 30, 40, 50, 60, 70, 80, 90, 100 or more different proteoforms of a certain type of protein of interest present in a sample, in order to provide a more comprehensive proteoform profile for that protein in that particular sample. As noted elsewhere herein, these different proteoforms may include any of a number of truncated species, species with amino acid post translational modifications (PTM) of any number (1, 2, 3, 4, 5, 6, 7, 8, 9, 10 or more) different types of PTMs as described above (e.g., phosphorylation, ubiquitylation, acylation, methylation, nitration, etc.), and a proteoform may include 1 PTM, 2 PTMs, 3 PTMs, 4 PTMs, 5 PTMs, 6 PTMs, 7 PTMs, 8 PTMS, 9 PTMs, 10 PTMs, 11 PTMs, 12 PTMs, 13 PTMs, 14 PTMs, 15 PTMs, 20 PTMs, or more. These PTMs may exist in full-length or as noted previously, in truncated versions of the protein species of interest.

These comprehensive proteoform profiles can then be analyzed with relative to other samples to ascertain differences between those samples, e.g., healthy vs. diseased samples, changes to a patient over time, changes to the profile following treatment or administration with potential therapeutics or other analytes, timing of changes in samples from the same biological system relative to disease onset, progression, and/or phenotypic presentation of, e.g., symptoms, and the like.

For example, a sample may be analyzed to identify all of the different proteoforms of a given protein of interest that are present in that sample, as well as each form's relative abundance. This may include identification of the presence or absence, as well as relative abundance when detected as present, of proteoforms that reflect the full range of possible combinations of the modifications set forth herein. Once a profile has been generated for a given sample, the mere presence of specific proteoforms, the relative abundance of different proteoforms within that sample, and/or the level of molecular heterogeneity reflected by the proteoform profile of that sample may be compared to other samples to ascertain differences.

For example, in some cases, samples may be obtained from healthy patients or biological systems and their proteoform profiles compared against samples derived from diseased patients or systems, to identify changes in those profiles in order to ascertain potential biological pathways leading to those changes. Such samples may come from the same patient or system and taken before and after disease onset, or from different patients or systems who reflect healthy and diseased states.

Likewise, in some cases, samples may be obtained from patients or model systems that that include those that are and are not treated with potential effectors of biological functions or pathways that are believed to impact disease onset, progression or potential remediation, to identify potential pathway triggers and potential interventions to arrest or remediate disease onset and/or progression. Again, such samples may be from the same patient or system or from different patients or systems that reflect treatment and non-treatment.

In addition, in some cases, samples may be analyzed from a given patient or system over time, or among multiple patients at different points in disease progression, to ascertain how the proteoform profiles may change over time, what aspects may reflect transitional events for onset of a disease or transition to different phases of a disease, treated vs. not treated with potential effector compounds or protocols, samples from systems that are treated over time, to analyze time courses of changes, samples that are derived from systems at differing times relative to disease onset and progression, e.g., to ascertain threshold or transitional events in disease onset and progression, and the like. By identifying the changes in proteoform profiles that occur at specific junctures of disease onset and progression, one can better pinpoint causative events and/or conditions that lead to such transitions. By knowing such causes, one can better assess potential interventions to block, arrest or significantly impede disease progression, which can, in turn, be tested and evaluated using the above-described processes.

As noted repeatedly above, reference to samples includes samples that may be patient derived or derived from simple (e.g., simple in vitro systems) or complex model systems (mammalian models such as mice, organoids, miBrains, etc.), as described elsewhere herein, and may include cells, cell lysates, purified proteins, tissues from, e.g., brains, blood, plasma, cells from blood, cerebral spinal fluid (CSF), or any relevant source for such samples.

### G. Reagents

As alluded to above, the present disclosure provides for the various reagents used in the herein described methods and systems. For example, included herein are affinity reagents, and combined libraries of affinity reagents that have relatively high affinity for specific characteristics of different proteoforms of a given protein of interest. These reagents may include antibodies, antibody fragments, aptamers, binding proteins, binding peptides, or the like that are capable of specifically binding to a given characteristic of a proteoform of the protein of interest. In particularly preferred aspects, the affinity reagents may include detectably labeled antibodies or binding fragments of antibodies, such as fluorescently labeled antibodies. For proteoform analysis, such libraries may include 2, 3, 4, 5, 6, 7, 8, 9, 10, 15, 20, 25, 30, 35, 40, 45, 50, 60, 70, 80, 90, 100 or more different affinity reagents that have binding specificity for different characteristics of proteoforms for each different protein of interest for which proteoform analysis is desired. In some cases, the libraries may include reagents that target 1, 2, 3, 4, 5, 6, 7, 8, 9, 10, 15, 20, 25, 30, 35, 40, 50, 100, 500 or more different proteins of interest and their respective proteoforms. These libraries are typically stored in multi-well plates or other similar storage vessels where each different reagent, or set of reagents, is separately stored from each other. In some cases, multiple different reagents may be stored within the same reagent vessel or storage component thereof, where they may be differentiated during detection, e.g., through detectably different fluorescent labels attached to the different reagents, e.g., different fluorescent labels having different emission spectra or other optical characteristics.

For purposes hereof, the affinity reagents useful in performing particular analyses for proteoforms may typically include affinity reagents that bind specifically to specific forms of the protein or proteins of interest, e.g., bearing specific post translational modifications, or for regions of the protein(s) of interest that may be lacking in certain splice isoforms of the protein. Such affinity reagents may, in many cases, be acquired from commercial sources where available, e.g., Abcam PLC, and/or Cell Signaling Technology, Inc. Alternatively, generation of affinity reagents, e.g., antibodies, antibody fragments etc., may be generated using known techniques, including, for example, polyclonal and monoclonal antibody generation methods generated against polypeptides representing the particular epitope of interest, phage display Fab generation methods, and the like.

By way of example, reagent libraries for use in analyzing isoforms and proteoforms of the various different proteins of interest described herein may include antibodies specific for each of the various characteristics of the above described isoforms and proteoforms, including for example, antibodies that are specific for epitopes within such proteins that include the individual modifications, or that are specific for segments of the protein that are lacking in in any N-terminal or C-terminal truncations or other splice variations, above, as well as segments that are present in all isoforms as a control. In particular, such libraries may include affinity reagents that are specific for individual phosphorylation, nitration, acetylation, ubiquitylation or other modified sites in these different proteoforms of each of the proteins of interest, including those set forth in Tables 1 through 7, above.

As noted above, variety of affinity reagents specific for the above described proteins of interest, and their modified forms are commercially available from e.g. Abcam PLC, Cell Signaling Technology, Inc., and may be configured for use in the methods and processes described herein, e.g., through attachment of detectable labels, etc. Alternatively, a number of conventional antibody generation techniques may be employed to produce such antibodies, including targeted immunogenesis followed by differential screening, screening of Fab phage display libraries for specific binders, and the like. In particular, proteins or shorter peptide fragments of proteins bearing a given modification at a given site may be used to generate and/or screen for antibodies or other affinity reagents that are capable of binding a specifically modified site in a protein of interest.

In certain aspects, the methods and affinity reagent libraries or panels described herein may include at least 2, at least 3, at least 4, at least 5, at least 6, at least 7, at least 8, at least 9, at least 10, at least 11, at least 12, at least 15, at least 20, at least 30, at least 40, at least 50, at least 60, at least 70, at least 80, at least 90, at least 100, or more than 100 of the aforementioned affinity reagents that are capable of differentially binding to different modifications to or isoforms of the protein of interest. In some cases, the reagent libraries or panels may include affinity reagents capable of differentially binding to different modifications or isoforms of the protein of interest, including up to 100 different affinity reagents, up to 50 different affinity reagents, up to 30 different affinity reagents, up to 25 different affinity reagents, up to 20 different affinity reagents, or up to 10 different affinity reagents.

### H. Kits

In addition to the foregoing reagents, also provided herein are kits useful in carrying out the analyses described herein, which kits may include the affinity reagents described above, along with one or more of the enrichment reagents used to enrich for low abundance proteins and proteoforms, e.g., beads and antibodies used for the immune-isolation and/or immunoprecipitation of the proteins of interest, wash and other elution reagents, for such enrichment standard proteins or polypeptides, and the like. Such kits may also include the flow-cells and arrays used to immobilize proteins of interest in a single molecule, optically detectable format for subsequent analysis in appropriately configured optical detection systems described below. Such kits will typically include instructions for carrying out the enrichment, flow-cell deposition, interrogation and follow on analysis of biological samples using such kits.

### I. Systems

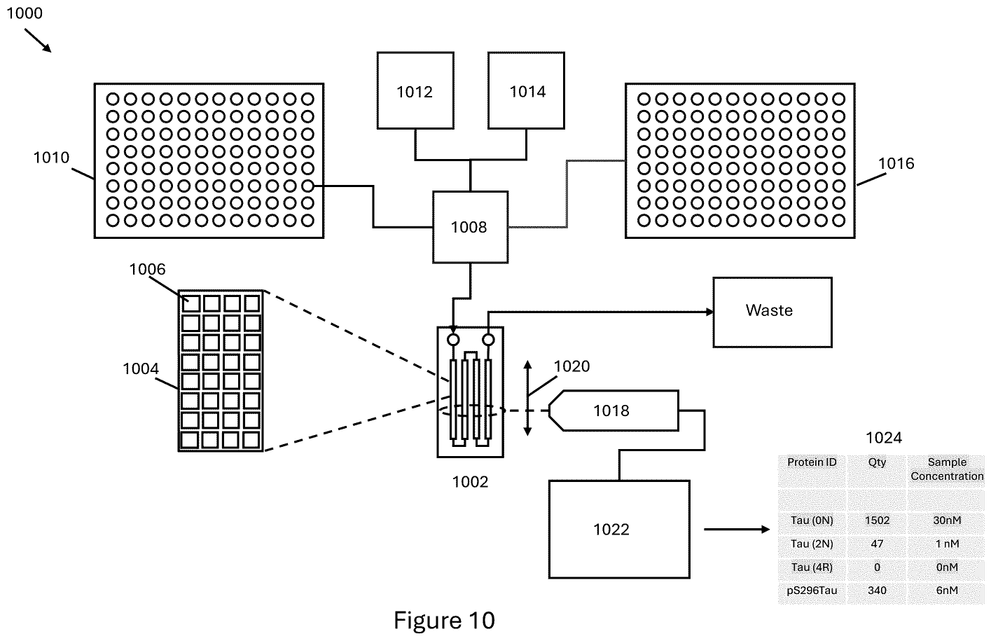

As also noted above, provided herein are systems for carrying out the analyses of different proteoforms of proteins of interest in biological samples. An example of such a system is illustrated in FIG. 10. As shown, the system 1000 includes a flowcell 1002 that includes one or more array surfaces (shown as 1004) within the separate channels or lanes of the flow cell upon which individual protein molecules from a sample may be deposited and immobilized in locations 1006 that are individually addressable, and in particular cases are individually optically resolvable from each other using, e.g., fluorescence microscopy or scanning techniques. In some cases, different lanes may include proteins of interest from different samples, different controls (e.g., null or control standard lanes, as described above), different treatments, etc.

The system will also typically include a fluidic delivery system 1008 that is configured to deliver different fluids to the flow cell 1002 through a series of fluidic lines and utilizing appropriate pumps, valves and other conventional fluid controls. The fluidics system 1008 may be fluidically coupled to various sources of fluids and reagents needed to carry out the analysis on the flow cell. For example, as shown, fluidic system 1008 is fluidly coupled to a source of a plurality of reagents 1010 (shown as a 96 well plate, although any number of different reagent storage systems of varying capacity may be employed) that includes a library of multiple affinity reagents that each have affinity for different characteristics of proteoforms of one or more proteins of interest. In certain aspects, the reagent sources include reagent libraries or panels that are fluidically coupled to the fluidic system 1008 may include a panel of antibodies that specifically recognize and bind to particular proteins or proteins of interest, including, for example, the affinity reagents described above for analyzing Tau proteoforms. In certain particularly preferred aspects, the systems described herein may include reagent panels that are fluidically coupled to the fluidic system, and in many cases, thereby coupled to the flow cells described above, that include at least 2, at least 3, at least 4, at least 5, at least 6, at least 7, at least 8, at least 9, at least 10, at least 11, at least 12 or more, 15 or more, 20 or more, 30 or more, 40 or more, 50 or more, 60 or more, 70 or more, 80 or more, 90 or more, or 100 or more of the aforementioned affinity reagents that are capable of differentially binding to different modifications to or isoforms of the Tau protein. In some cases, the reagent libraries or panels may include affinity reagents capable of differentially binding to different modifications or isoforms of the Tau protein, including up to 100 different affinity reagents, up to 50 different affinity reagents, up to 30 different affinity reagents, up to 25 different affinity reagents, up to 20 different affinity reagents, or up to 10 different affinity reagents.

The fluidic system 1008 may also be coupled to sources of washing fluids or buffers 1012, and removal reagents 1014 (for removing bound affinity reagents following detection), as well as any other ancillary fluids and reagents needed for the analysis. Similarly, where flow cells are prepared on the system, the fluidic system may be coupled to sources of different sample materials that are to be analyzed 1016 (again, shown as a 96 well plate, although again, any suitable sample storage system or capacity may be suitable).

The reagent sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, U.S. Patent Application No. WO 2023/122589A2, the full disclosure of which is hereby incorporated herein by reference in its entirety for all purposes.

The systems described herein also typically include a detection system, such as optical detection system 1018, for detecting and recording fluorescent signals arising from different positions on the array surface. Such detection systems may generally include line scanning confocal fluorescent microscope systems, which are capable of scanning across large array surfaces (as shown by arrow 1020) to detect and record fluorescence across such surfaces at reasonably high scan rates.

The overall systems also typically include one or more computers or processors 1022 for controlling the operation of the instrument system including the fluidic system 1008 (e.g., to sample different sample sources 1016, reagent sources 1010 and delivery timing and volume of each), and detection system 1018, among other functions, and for recording the detected signals received from the detection system 1018, e.g. fluorescent signals, and analyzing such signals to identify potential binding by each of the different affinity reagents. Included in such processors 1022 may be bioinformatic software or firmware that evaluates the signals received and based upon appropriate modeling, identifies likely positive binding events, and then subsequently provides an overall assessment of which proteoforms are present at any given location on the array as well as the relative abundance of each different proteoform across the array and ultimately, within the sample being analyzed, e.g., as shown at 1024. Examples of bioinformatic software processes for analyzing such proteoform and proteome data have been describe in, for example, U.S. Pat. Nos. 11,545,234, 10,473,654B1, and Eggertson, et al., bioRxiv, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud based systems, which house the informatics software that performs this latter analysis and reporting.

### VI. Examples

### Proteoform Analysis

The methods, processes, systems, devices and reagents described herein may generally be employed to characterize different proteoforms of proteins present in biological samples, as set forth in the following hypothetical example. As set forth above, biological samples may include any of a variety of biologically relevant samples, including those from patients, model systems or the like, and may include purified proteins, cell lysates, tissue samples, and may be obtained from brain, cerebral spinal fluid, blood, plasma, cells in blood, and the like.

In these processes, sample polypeptides or proteins to be analyzed that include at least a subpopulation of the protein of interest are coupled to structured nucleic acid particles (or SNAPs) that comprised a DNA origami framework with a single point of attachment for the protein or polypeptide. These structures are then deposited on a surface of a patterned flow-cell, such that individual protein/SNAP structures will be separate and optically resolvable from each other deposited protein/SNAP structure. The flow cells are then placed into an instrument that iteratively delivers different fluorescently labeled affinity reagents, e.g., antibodies, specific for different characteristics of the various different isoforms and proteoforms of the protein of interest, e.g., different phosphorylation sites, different ubiquitylation sites, etc., with intervening wash cycles and fluorescent detection cycles to identify where on the array the various affinity reagents would bind. In the case of the example protein, a number of modification sites are to be analyzed, including phosphorylations at tyrosine residues 39 and 125 and serine residue 129.

Following sample loading on the array, the arrays are then washed and then iteratively interrogated with affinity reagents. For example, in a first step, the proteins immobilized on the array are interrogated with a first affinity reagent or set of affinity reagents that specifically binds to the protein of interest, regardless of its modification form. The locations on the array that are bound by the affinity reagents in this first step are then identified as locations on the array at which the various different sub-species of the protein of interest are immobilized.

In subsequent iterative steps, following a wash step to remove previously bound affinity reagents, additional affinity reagents are contacted with the array that have binding specificity for differently modified forms of the protein of interest, such as phosphorylation or ubiquitylation sites.

In each affinity reagent interrogation step, those locations on the array at which a molecule of a hypothetical protein of interest was previously identified are examined to determine if the modification-specific affinity reagent binds, and the binding information is aggregated. As shown in FIG. 11, a hypothetical output of that analysis shows the detection of each different form of the protein of interest that reflects each type of interrogated modification. Briefly, as shown in panel A, the binding data reflects which proteoforms that are characterized by the interrogated modifications are present on the array, e.g., which individual protein molecules on the array possess each individual modification (e.g., only pY39), as well as which include more than one single modification among those interrogated, e.g., all of pY39, pY125 and pS129, or subset thereof, or where none of the modifications are present in the protein of interest. Once the different molecules are characterized as to their proteoform, one may quantify the different forms present on the array to provide a relative quantity of each form in the sample (see FIG. 11, Panel B). As shown in the hypothetical example, proteins of interest that include none of the analyzed modifications represent the predominant species of the protein in the sample, while those that are phosphorylated at 125 residue represent the next most prevalent species, followed by triple phosphorylated species, the subspecies phosphorylated only at serine 129, the subspecies modified only at tyrosine 39, and then those that are double phosphorylated at tyrosines 39 and 125, and tyrosine 125 and serine 129. The foregoing example is provided merely for illustration and is not intended to represent any biologically relevant proteoform characterization.

Analyses of the type set forth above may be used to interrogate samples from both healthy and diseased patients, and or from patients before and after treatment with existing or experimental drug candidates, in order to identify changes in their proteoform over time or in response to treatments. By way of example, the methods, processes, systems, reagents and devices described herein may be used to assess the various proteoforms of a protein of interest that are present in a sample. This analysis can be comprehensive, e.g., assessing all proteoforms present, or it can be targeted, e.g., to assess the presence and quantities of specific proteoforms. Such analyses allow assessment of the relative abundance of different isoforms, post translational modification occupancy, different truncations or splice isoforms, etc. By performing such analyses, one can explore changes or shifts in any of these aspects of those proteoforms over time, in healthy versus diseased patient samples, in model systems used to visualize and model the progression of different pathologies, and in response to exposure to treatments, therapies or other external influences.

While preferred embodiments of the present invention have been shown and described herein, it will be understood to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of analyzing proteins in a first sample, comprising:
providing a population of individual protein molecules from the first sample wherein said individual protein molecules are individually addressable, and wherein the population of individual molecules comprises a plurality of individual molecules of at least one protein of interest selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase 2 (LRRK2), RAC-alpha serine/threonine protein kinase (AKT1), and Mothers against decapentaplegic homolog 2 protein (SMAD2);
identifying a proteoform of the at least one protein of interest represented by each of the plurality of individual molecules of the at least one protein of interest based upon identification of a presence or absence of at least 3 different modifications within each of the individual molecules of the at least one protein of interest; and
characterizing a plurality of proteoforms of the at least one protein of interest present in the sample.

2. The method of claim 1, wherein the at least one protein of interest comprises catenin beta 1 protein.

3. The method of claim 2, wherein the at least 3 different modifications of beta catenin 1 protein are selected from the modifications set forth in Table 1.

4. The method of claim 1, wherein the at least one protein of interest comprises ERK2 protein.

5. The method of claim 4, wherein the at least 3 different modifications of ERK2 protein are selected from the modifications set forth in Table 2.

6. The method of claim 1, wherein the at least one protein of interest comprises EGFR protein.

7. The method of claim 6, wherein the at least 3 different modifications of EGFR protein are selected from the modifications set forth in Table 3.

8. The method of claim 1, wherein the at least one protein of interest comprises HER2 protein.

9. The method of claim 8, wherein the at least 3 different modifications of HER2 protein are selected from the modifications set forth in Table 4.

10. The method of claim 1, wherein the at least one protein of interest comprises LRRK2 protein.

11. The method of claim 10, wherein the at least 3 different modifications of LRRK2 protein are selected from the modifications set forth in Table 5.

12. The method of claim 1, wherein the at least one protein of interest comprises AKT1 protein.

13. The method of claim 12, wherein the at least 3 different modifications of the AKT1 protein are selected from the modifications set forth in Table 6.

14. The method of claim 1, wherein the at least one protein of interest comprises SMAD2 protein.

15. The method of claim 14, wherein the at least 3 different modifications of SMAD2 protein are selected from the modifications set forth in Table 7.

16. The method of claim 1, wherein the identifying step comprises identifying a presence or absence of at least 5 different modifications to each of the individual molecules of the at least one protein of interest.

17. The method of claim 16, wherein the identifying step comprises identifying a presence or absence of at least 7 different modifications to each of the individual molecules of the at least one protein of interest.

18. The method of claim 17, wherein the identifying step comprises identifying a presence or absence of at least 10 different modifications to each of the individual molecules of the at least one protein of interest.

19. The method of claim 1, wherein the population of individual protein molecules are immobilized on individually addressable locations of an array surface.

20. The method of claim 1, wherein the first sample comprises at least 5 different proteoforms of the at least one protein of interest.

21. The method of claim 20, wherein the first sample comprises at least 20 different proteoforms of the at least one protein of interest.

22. The method of claim 1, wherein the identifying step is configured to identify at least 5 different proteoforms of the at least one protein of interest.

23. The method of claim 22, wherein the identifying step is configured to identify at least 20 different proteoforms of the at least one protein of interest.

24. The method of claim 23, wherein the identifying step is configured to identify at least 100 different proteoforms of the at least one protein of interest.

25. The method of claim 1, further comprising a step of quantifying an amount of each of the plurality of different proteoforms of the at least one protein of interest in the first sample characterized in the characterizing step.

26. The method of claim 1, wherein identifying the presence or absence of modifications within each individual molecule of the at least one protein of interest comprises:
contacting the individual molecules of the at least first protein of interest with a plurality of affinity reagents, wherein each of the plurality of affinity reagents comprises a specific binding affinity for a different modification to the at least one protein of interest; and
detecting whether each of the plurality of affinity reagents binds to individual molecules of the at least one protein of interest.

27. The method of claim 1, further comprising repeating the providing, identifying and characterizing steps with a population of individual protein molecules from a second sample that comprises a plurality of molecules of the at least one protein of interest, and comparing proteoforms of the at least one protein of interest characterized from the first sample to proteoforms of the at least one protein of interest characterized from the second sample.

28. The method of claim 1, wherein the providing, identifying and characterizing steps are repeated with a population of individual protein molecules from at least 10 different samples.

29. The method of claim 1, wherein the providing, identifying and characterizing steps are repeated with a population of individual protein molecules from at least 50 different samples.

30. The method of claim 1, wherein the providing, identifying and characterizing steps are repeated with a population of individual protein molecules from at least 100 different samples.

31. The method of claim 1, wherein the providing, identifying and characterizing steps are repeated with a population of individual protein molecules from at least 1000 different samples.

32. The method of claim 1, wherein the population of individual molecules comprises a plurality of individual molecules of at least a second protein of interest, and the identifying and characterizing steps further comprise identifying and characterizing proteoforms of the second protein of interest.

33. A system for characterizing proteins, comprising:
one or more solid supports comprising molecules of at least one protein of interest immobilized thereon, wherein the at least one protein of interest is selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2) proteins, and wherein individual molecules of the at least one protein of interest are individually addressable;
a source of a plurality of different affinity reagents, each different affinity reagent having a binding affinity to the at least one protein of interest having a different modification;
a fluidic system for delivering the plurality of different affinity reagents to the one or more solid supports to contact the affinity reagents with the individual molecules of the at least one protein of interest;
a detector for detecting whether each of the different affinity reagents binds to individual molecules of the at least one protein of interest;
a processor programed to characterize proteoforms of the at least one protein of interest present on the one or more solid supports from detected binding or nonbinding of the different affinity reagents to the individual molecules of the at least one protein of interest.

34. The system of claim 33, wherein the plurality of different affinity reagents comprises affinity reagents that specifically bind molecules of the at least one protein of interest having one or more of the modifications set forth in one of Tables 1 through 7.

35. The system of claim 33, wherein the one or more solid supports comprises an array surface disposed within a flowcell, wherein the individual molecules of the at least one protein of interest are immobilized on the array surface at individually addressable locations.

36. The system of claim 33, wherein the detector comprises a laser induced fluorescence detector, and wherein the different affinity reagents each comprise a fluorescent label.

37. The system of claim 35, wherein the array surface comprises at least 10,000 individual protein molecules immobilized on the array surface at individually addressable locations.

38. The system of claim 33, wherein the processor is further programmed to quantify an amount of each proteoform of the at least one protein of interest characterized as present on the one or more solid supports.

39. An array, comprising:
a plurality of individual molecules of at least one protein of interest deposited on a surface of the array and positioned to be individually addressable, wherein the at least one protein of interest is selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2), and wherein, and wherein the plurality of individual molecules of the at least one protein of interest comprises at least two proteoforms of the at least one protein of interest; and
a first affinity reagent having binding specificity for at least a first characteristic of at least one of the two proteoforms of the at least one protein of interest, the first affinity reagent being bound to individual molecules of the at least one protein of interest possessing the first characteristic of at least one of the two proteoforms of the at least one protein of interest.

40. A library of reagents, comprising:
a plurality of sources of affinity reagents, where each source of the plurality of sources contains a separate affinity reagent; and wherein each affinity reagent:
has a binding specificity for a different characteristic of one or more proteoforms of at least one protein of interest selected from catenin beta 1, mitogen activated protein kinase 1 (ERK2), epidermal growth factor receptor (EGFR), receptor tyrosine kinase erbB-2 (HER2), leucine rich repeat serine/threonine-protein kinase protein 2 (LRRK2), RAC-alpha serine/threonine protein kinase (AKT1), and Mothers against decapentaplegic homolog 2 protein (SMAD2); and
a detectable label attached to the affinity reagent.
